(19) United States
(12) Patent Application Publication
(10) Pub. No.: US 2010/0294927 A1
(43) Pub. Date: Nov. 25, 2010

(54) High throughput inspecting

(75) Inventors: Michael Nelson, Libertyville, IL (US); Sylvain Cruchon-Dupeyrat, Bellevue, WA (US); Bjoern Rosner, Chicago, IL (US); Cedric Loiret-Bernal, Evanston, IL (US)

Correspondence Address: FOLEY AND LARDNER LLP;SUITE 500, 3000 K STREET NW, WASHINGTON, DC, 20007, US

(73) Assignee: Nanolnk, Inc.

(21) Appl. No.: 11/519,199

(22) Filed: Sep. 12, 2006

Related U.S. Application Data
(60) Provisional application No. 60/715,583, filed on Sep. 12, 2005.

(51) Int. Cl. G01N 23/00; A61K 9/44; G06F 19/00; G01N 1/02

(52) U.S. Cl. 250/307; 424/10.2; 250/311; 702/22; 73/864.81; 73/863.03

(57) ABSTRACT

The various embodiments provide methods and apparatus high-throughput reading and decoding of information-encoding features (especially identification features) on pharmaceutical compositions for the purpose of e.g. counterfeiting detection and inventory tracking/tracing. A preferred embodiment provides high-throughput imaging of regular arrays of pharmaceutical tablets with a scanning electron microscope. Another preferred embodiment provides video-rate scanning probe imaging of pharmaceutical compositions and especially atomic force microscopy imaging thereof.

### RELATED APPLICATIONS

This application claims priority to U.S. Provisional application Ser. No. 60/715,583 filed Sep. 12, 2005, which is hereby incorporated by reference in its entirety.

### BACKGROUND

The various embodiments described herein generally relate to counterfeiting and illegal trading detection, and especially that of pharmaceutical tablets and capsules.

According to the World Health Organization (WHO), “fake pharmaceuticals account for as much of 10% (or $21 billion) of the global drug market [ . . . ] In some underdeveloped nations, as many of 40% of the drugs sold are counterfeit.” (“Trends in Security Packaging”, Pharmaceutical and Medical Packaging News, E. Swain, January 2003). Therefore, there is an acute commercial need for anti-counterfeiting solutions to detect fakes or pharmaceuticals illegally diverted from legitimate distribution channels. While package-level anti-tampering devices are known (including labels, radio-frequency identification tags, bar codes and chemical taggants), a need remains for unit-level counterfeiting protection, meaning the identification of individual tablets or capsules. This is especially important in the United States and in other countries where bulk distribution of pharmaceutical capsules and tablets through complex channels is prevalent.

Nanolnk, Inc. of Chicago, Ill. recently introduced a covert anti-counterfeiting technology that incorporates microscale and nanoscale identification patterns on the surface of e.g. a pharmaceutical tablet, capsule or wafer. The patterning technology may be based upon direct-write nanolithography, such as Dip Pen Nanolithography™ printing. Dip Pen Nanolithography and DPN™ are trademarks or registered trademarks of NanoInk, Inc. Said identification patterns may be extremely small in size, making them advantageously difficult to detect with the naked eye or with conventional optical inspection technology and even more difficult to duplicate. However, because they may be also much smaller than conventional labels, bar codes or any other indicia previously prepared on pharmaceutical units, standard reading and decoding techniques (such as laser scanning) do not apply.

Thus, there is a commercial need for methods and apparatus that locate, detect, read and/or decode microscale and nanoscale identification features or regions on objects and compositions, and especially pharmaceutical compositions. In particular, there is a need for techniques and apparatuses capable of locating, detecting, reading and/or decoding said identification regions with a high throughput, meaning for example more than 10 units/hour, and especially more than 100 units/hour, and especially more than 1,000 units/hour, and especially more than 5,000 units/hour.

There is a further need for (i) detecting the presence of said identification region(s) on pharmaceutical compositions under test to confirms their authenticity; and/or (ii) extracting information carried by said identification regions (such as a serial number) to trace said pharmaceutical composition to a given origin (batch number, manufacturing location, country, date of manufacture, expiration date . . . ) and/or track its progression in the distribution chain from the manufacturer to the end consumer. Given that a multiplicity of identification features may be fabricated in many locations of an object or composition (in a redundant manner or not), there is also a need for techniques and apparatus capable of decoding multiple identification regions in multiple locations.

The present document describes improvements upon (and further exemplification of) previously disclosed methods and apparatuses, as well as novel methods and apparatuses for the purpose of such detecting, reading and decoding.

No admission is made that any of the references cited in this Background or any other sections are prior art. All references are hereby incorporated by reference and can be referred to in the practice of the present invention.

Methods useful for high throughput operation in the semiconductor industry are not generally helpful for pharmaceutical processing. For example, alignment is a very important and challenging issue in semiconductor processing. Pharmaceutical may need different degassing or evacuation processes. Treatment of samples may be important for good results. Transitioning from batch to continuous or high speed processing may present challenges.

### SUMMARY

Exemplary embodiments of the present invention are summarized in this non-limiting section. Provided is, generally speaking, the detection of the presence or absence of substantially covert, micro- and nanoscale identification features imprinted on objects and compositions, and especially pharmaceutical compositions, identifying them as genuine or fake, or illegally traded. Read-out may occur at the distribution location (e.g. pharmacy) or preferably at a remote site. The site can be also highly secured. Co-imprinted overt features may be detected and analyzed as well.

One embodiment provides a method comprising: (i) providing a plurality of unit pharmaceutical compositions each having a surface to be inspected potentially comprising identification features; (ii) providing an inspection device which provides an inspection zone which allows inspecting the surface of the unit pharmaceutical compositions potentially comprising identification features; (iii) providing an introducing device which introduces the unit pharmaceutical composition into the inspection zone before inspection; (iv) providing a withdrawing device which withdraws the unit pharmaceutical composition from the inspection zone after inspection; (v) operating the inspection device, the introducing device, and the withdrawing device so that an inspection rate of unit pharmaceutical compositions is achieved of at least about 10 units per hour. The introducing device, the withdrawing device, or both can comprise a robotic device.

Another embodiment provides an apparatus comprising: at least one scanning electron microscope (SEM) comprising a first vacuum chamber for SEM inspection, and at least one second vacuum chamber which can be evacuated and filled separately from the first vacuum chamber, at least one first sample holder in the first vacuum chamber which is adapted to hold a plurality of sites for holding a plurality of unit pharmaceutical compositions to be inspected with the scanning electron microscope in the first vacuum chamber; at least one second sample holder in the second vacuum chamber which is adapted to hold a plurality of sites for holding a plurality of unit pharmaceutical compositions to be inspected with the scanning electron microscope in the first vacuum chamber; a sample holder transport device which transports the first sample holder from the first vacuum chamber to the second vacuum chamber; and the second sample holder from the second vacuum chamber to the first vacuum chamber, wherein the apparatus is adapted for a sample inspection rate of at least about 10 units per hour.

Also provided is a pharmaceutical composition comprising: a unit pharmaceutical composition comprising surface identification features which are adapted to be imaged by scanning electron microscopy; metallic coating over the identification features adapted to improve the imaging by scanning electron microscopy.

Also provided are methods and apparatus for the high-throughput imaging of identification features imprinted on objects and compositions and for the decoding of the information they may store. High-scan-rate scanning probe microscopy (and in particular atomic force microscopy), advanced optical inspection and especially scanning electron microscopy may provide high-throughput, high-resolution imaging. Methods and apparatus capable of imaging multiple objects or compositions simultaneously or in rapid succession are disclosed.

Also provided is a method for inspecting unit pharmaceutical compositions comprising:

providing a plurality of unit pharmaceutical compositions each having a surface to be inspected potentially comprising at least two identification features on each unit pharmaceutical;

providing a computer controlled scanning electron microscope inspection device which provides an inspection zone which allows inspecting the surface of the unit pharmaceutical compositions potentially comprising identification features;

providing an automated introducing device which introduces the unit pharmaceutical composition into the inspection zone before inspection;

providing an automated withdrawing device which withdraws the unit pharmaceutical composition from the inspection zone after inspection;

operating the inspection device, the introducing device, and the withdrawing device so that an inspection rate of unit pharmaceutical compositions is achieved of at least about 10 units per hour.

Also provided is a method for inspecting unit pharmaceutical compositions in high throughput mode with scanning electron microscopy comprising the combination of steps:

providing a plurality of unit pharmaceutical compositions each having a surface to be inspected potentially comprising at least two identification features on each unit pharmaceutical and adapted for SEM inspection;

providing a computer controlled scanning electron microscope inspection device which provides an inspection zone which allows inspecting the surface of the unit pharmaceutical compositions potentially comprising identification features;

providing an automated, robotic introducing device which introduces the unit pharmaceutical composition into the inspection zone before inspection;

providing an automated, robotic withdrawing device which withdraws the unit pharmaceutical composition from the inspection zone after inspection;

operating the inspection device, the introducing device, and the withdrawing device so that an inspection rate of unit pharmaceutical compositions is achieved of at least about 10 units per hour;

processing inspection data collected from the inspection device to determine whether identification features are present or absent.

Also provided is a method comprising:

providing a plurality of unit compositions or objects each having a surface to be inspected potentially comprising identification features;

providing an inspection device which provides the inspection zone which allows inspecting the surface of the unit compositions or objects potentially comprising identification features;

providing an introducing device which introduces the unit composition or object into the inspection zone before inspection;

providing a withdrawing device which withdraws the unit composition or object from the inspection zone after inspection;

operating the inspection device, the introducing device, and the withdrawing device so that an inspection rate of unit compositions or objects is achieved of at least about 10 units per hour.

Also provided are methods and apparatus for quality inspection at various steps during manufacturing of the encoded pharmaceutical composition. Working examples are provided.

### BRIEF DESCRIPTION OF THE DRAWINGS

FIG. 1 schematically illustrates the process of decoding a pharmaceutical composition having covert identification regions.

FIG. 2 is the schematic diagram of a dispensing unit that aligns and orients pharmaceutical units on a tray in preparation for high-throughput imaging.

FIG. 3 lists possible imaging techniques as a function of the throughput, resolution and cost.

FIG. 4 is a high-speed AFM image of five raised features.

FIG. 5 is a scanning electron micrograph of a set of identification features. B is a zoomed-in view of image A.

FIG. 6 is a scanning electron micrograph of one set of identification features produced using an environmental SEM setup.

FIG. 7 is a scanning electron micrograph of one set of identification features produced in two short acquisition times.

FIG. 8 illustrates schematically a scanning electron microscope that comprises a tray holding a large number of pharmaceutical units, permitting high-throughput imaging.

FIG. 9 illustrates the process for locating and imaging an identification region using successive optical and atomic force microscopy.

FIG. 10 is an optical micrograph of a superset of six sets of identification marks.

FIG. 11 is an atomic force microscopy image of an identification region.

FIG. 12 illustrates the steps of locating and imaging an identification feature, measuring some of its characteristics (e.g. the width of individual lines in the pattern, N=narrow, W=wide) to extract identification information (in this case, a binary string equivalent to a number or alphanumeric string) and comparing said information with a stored value in a database in order to identify the item under inspection as genuine or not.

FIG. 13 is the schematic diagram of the different components of a pharmaceutical tablet inspection instrument based upon a Scanning Electron Microscope.

FIGS. 14A, B, C, D and E illustrate a detailed, preferred algorithm to be run by the computing device part of the pharmaceutical tablet inspection instrument shown in FIG. 13.

FIG. 15 illustrates the decoding of a Data matrix barcode comprising a set of “dots” positioned at various locations of a grid as elements. The string encoded by the position of the dots was successfully decoded from the image that is shown.

FIG. 16 illustrates the decoding of a linear barcode comprising a set of “lines” of various widths as elements. In this case, the string encoded by the width sequence in the barcode was successfully decoded from the image that is shown.

FIG. 17 provides an example of data matrices.

FIG. 18 provides an example of a data matrices.

FIG. 19 provides an additional example of barcodes.

FIG. 20 provides an additional example of barcodes.

FIGS. 21 and 22 show in overt-covert imprinting a tablet and imprint area, wherein the logo is visible.

FIGS. 23 and 24 show in overt-covert imprinting covert imprints next to a logo.

FIGS. 25 and 26 show in overt-covert imprinting imprints wherein the barcodes are raised; they protrude from the surrounding surface. The barcode line and spacing target width are 100 nm and 200 nm.

FIGS. 27 and 28 show in overt-covert imprinting comparison of two bar codes in the same array. The left one has some beam damage from SEM imaging in its lower right corner.

FIGS. 29 and 30 show in overt-covert imprinting barcodes having line and spacing target width of 100 and 200 nm. In this imprint, the barcodes are recessed into the surface.

### DETAILED DESCRIPTION

References will be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims. All references in this specification are incorporated by reference in their entirety and can be relied upon in general in practicing the invention.

Priority U.S. Provisional application Ser. No. 60/715,583 filed Sep. 12, 2005 is hereby incorporated by reference in its entirety including claims and figures.

### High-Throughput Inspection Process

In a first embodiment, provided is a method for high-throughput processing of objects and compositions, and especially pharmaceutical compositions, for detecting counterfeits. The method comprises the steps of (1) preparing a sample of said objects and compositions for inspection; (2) inspecting said objects and compositions to (a) determine the presence or absence of substantially covert, micro- and nanoscale identification features printed or imprinted on said objects and compositions or (b) decode the information stored in said identification features (in the form of spatial or depth encoding, for example), if any; and (3) utilizing said determination and said information to identify said objects or compositions as genuine, fake or illegally traded.

The micro- and nanoscale identification features are not particularly limited by any shape and can be, for example, dots, circles, lines, rectilinear structures, curvilinear structures, or bar codes, whether linear or radial or data matrices. Other examples include geometric objects such as, for example, triangles or rectangles. The identification features can be space filling such as, for example, a disk or can be non-space filling such as, for example, a donut or circle with a hollowed out interior. The identification features can be, for example, periodic arrays of lines or dots. The identification features and structures, such as barcodes, can also have specific positions relative to one another

The rate of processing of said objects and compositions may be 10 units per hour or more and especially 100 units per hour or more, or more particularly, 250, 500, or 750 or more, and especially 1,000 units per hour or more, or more particularly, 2,000, 3,000, or 4,0000 and more, and especially 5,000 units per hour or more.

The preparation step may comprise sampling a large collection of said objects and compositions to reduce the number of items to a manageable amount; and preparing said sample for introduction into an imaging member.

The inspection step comprises at least one (high-resolution) scanning or imaging step, using said imaging member, which can be in a non-limiting way a scanning probe microscope, a scanning electron microscope, or a system comprising an optical microscope and a video capture system, or a combination thereof, to obtain an image or cross-section of said micro- to nanoscale identification features on said objects and compositions.

The inspection step may also comprise the step of locating said micro- to nanoscale identification regions using at least one coarse-resolution inspection member adapted to generate coarse-resolution inspection data. A single inspection instrument may in some cases provide both low- and high-resolution imaging steps or two instruments may be integrated into one system.

The step of determining the presence or absence of substantially covert, micro- and nanoscale features from the results of the inspection step(s) may comprise utilizing a computing device loaded with an algorithm adapted to recognize patterns from said coarse- and high-resolution inspection data and judge of the presence or absence of said identification features with the help of an optional information store.

The step of decoding the information stored in said identification features (in the form of spatial or depth encoding, for example), if any, from the results of the inspection step(s) may comprise utilizing a computing device loaded with an algorithm adapted to recognize patterns from said coarse- and high-resolution inspection data, measure one or more of its characteristics of said identification features, extract identification information from said characteristics with the help of additional data storage and a set of rules (e.g. describing how said identification features were designed in the first place) or algorithms, and categorize said identification information with the help of an optional information store.

The step of identifying said objects or compositions as genuine, fake or illegally traded comprise utilizing (i) the information in the previous two steps and (ii) an additional data storage e.g. comprising data linking said identification information to the manufacturing and distribution information of said object or composition and its expected market, to yield information about the unit's origin or path through distribution channels and finally identify said objects or compositions as genuine, fake or illegally traded (found in a market where it should not be present).

Preferably, care is taken throughout the high-throughput object inspection process to create a verifiable, auditable data trail via proper recording of each step and procedures, tracking of each pharmaceutical unit or unit group throughout the inspection facility e.g. with the help of container bar code and RFID labeling, use of automated laboratory management methods and software, and automated reporting.

In a variation of the described embodiments, pharmaceutical units with at least two different kinds of information bearing marks are inspected. There is a larger type of information-bearing marks, called overt features, that have dimensions in X and Y (in plane) of many micrometers. These features may be read with some of the lower-resolution techniques described above. The other type of information bearing marks is smaller and can not be readily resolved with a normal optical microscope. These features are called covert features. These covert features can furthermore appear in overt or covert groups. Information about the relative placement of said overt and said covert marks may be used to facilitate the step of locating said covert micron- to nanometer-scale features.

### Sampling and Preparation Steps

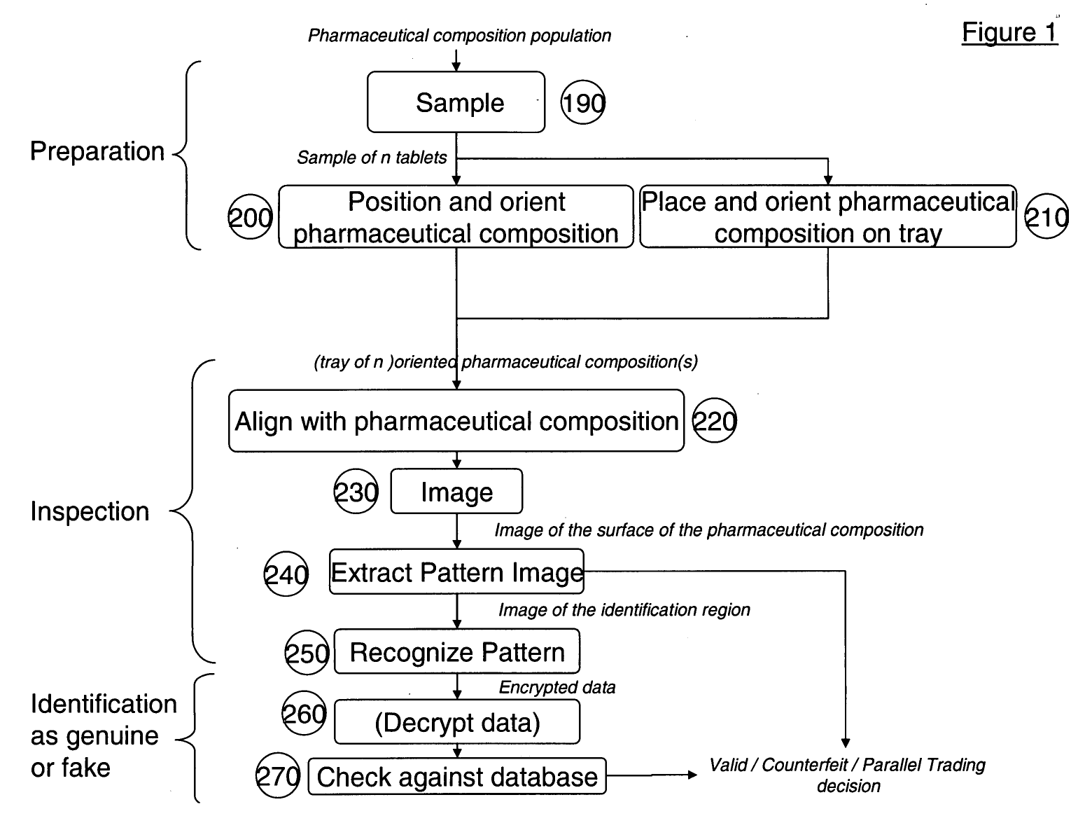

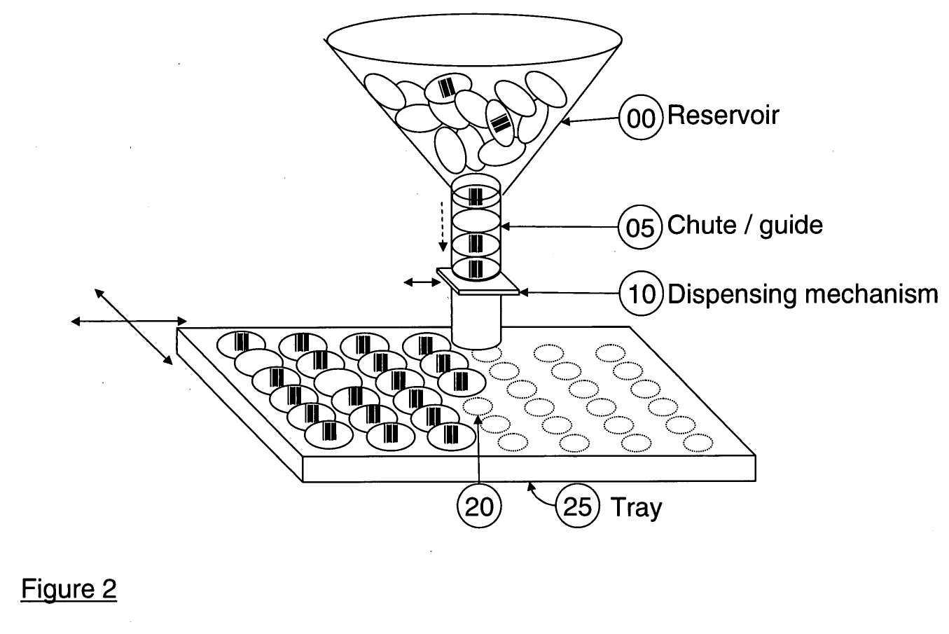

FIGS. 1 and 2 are an example of the methods described and claimed herein. The figures illustrate unit pharmaceutical compositions, each having a surface to be inspected potentially comprising identification features.

First, at least one unit is placed and oriented on a sample holder (1-200). In a preferred embodiment, many units are placed and oriented next to each other in known locations on a tray (1-210). In a further preferred embodiment, a tray (2-25) that contains pre-determined place holders for the pharmaceutical units (2-20) is placed underneath a dispensing machine. The dispensing machine may comprise: a reservoir for the pharmaceutical units (2-00), a unit sorting and guiding subsystem (2-05) and a dispensing mechanism (2-10). In operation, the tray or dispensing instrument moves relative to each other, so that the dispensing mechanism and an unfilled place holder on the tray are in registration, dispenses a unit, preferably with a fixed orientation compared to other already-dispensed units, and then moves to the next location.

Another embodiment comprises one or more persons such as technicians manually positioning and orienting the pharmaceutical composition for imaging such as place and orient on a tray. A combination of automatic and manual methods can be also done. The positioning and placement is carried out so that the surface to be imaged is properly exposed for imaging.

Another embodiment provides a cover for the tray to better protect the contents for storage, security, or transport. For example, the tray and cover could be adapted so that a person may trip and yet not upset the contents of the tray.

The individual sites on the tray can be indexed for better identification. The tray can be adapted for specific drugs and dosages.

Also provided is an inspection device such as a reading instrument which provides an inspection zone which allows inspection of the surface of the unit pharmaceutical compositions. The filled tray or sample holder can be then aligned to the reading instrument (2-220). For example, a staging system automatically positions the tablets for imaging. The reading instrument then images the identification region (2-230). A low magnification image of the imprint area can be acquired to identify the overt alignment features. High magnification images of individual barcode patterns can be then acquired. From these images, the pattern of interest can be detected and extracted (2-240). Its presence or absence alone can in one embodiment be used to form a valid/counterfeit decision. Furthermore, image processing and pattern recognition algorithms can be used to extract encoded information from these images.

The extracted encoded information may have to be further processed to be recognized as encrypted data (2-250). The encrypted data is then decoded (2-260) to yield accessible data that is then checked against the contents of at least one database (2-270). This may result in a decision regarding valid/counterfeit or parallel trading status of the pharmaceutical unit.

The database can be from, for example, a company running the authentication process or also a database provided by a pharmaceutical company. For example, the pharmaceutical company may know the intended geographic distribution of their goods. Hence, the method can further comprise the step of identifying the unit pharmaceutical with use of a pharmaceutical data base comprising geographical distribution.

The introducing device can be robotically controlled, or it can be a computer controlled, automated device.

A withdrawing device can be also provided which withdraws the unit pharmaceutical composition from the inspection zone after inspection. Withdrawing can be executed manually or robotically. It can comprise a computer controlled, automated device.

The introducing device and the withdrawing device can be operatively coupled with the particular inspection device and operative coupled with each other to provide for high throughput processing. For example, the introducing device can place a tray into the inspection zone, whereas the withdrawing device can then remove the tray from the inspection zone. A single device can be used as needed which is adapted to both introduce as well as withdraw the items to be inspected.

### Imaging Techniques

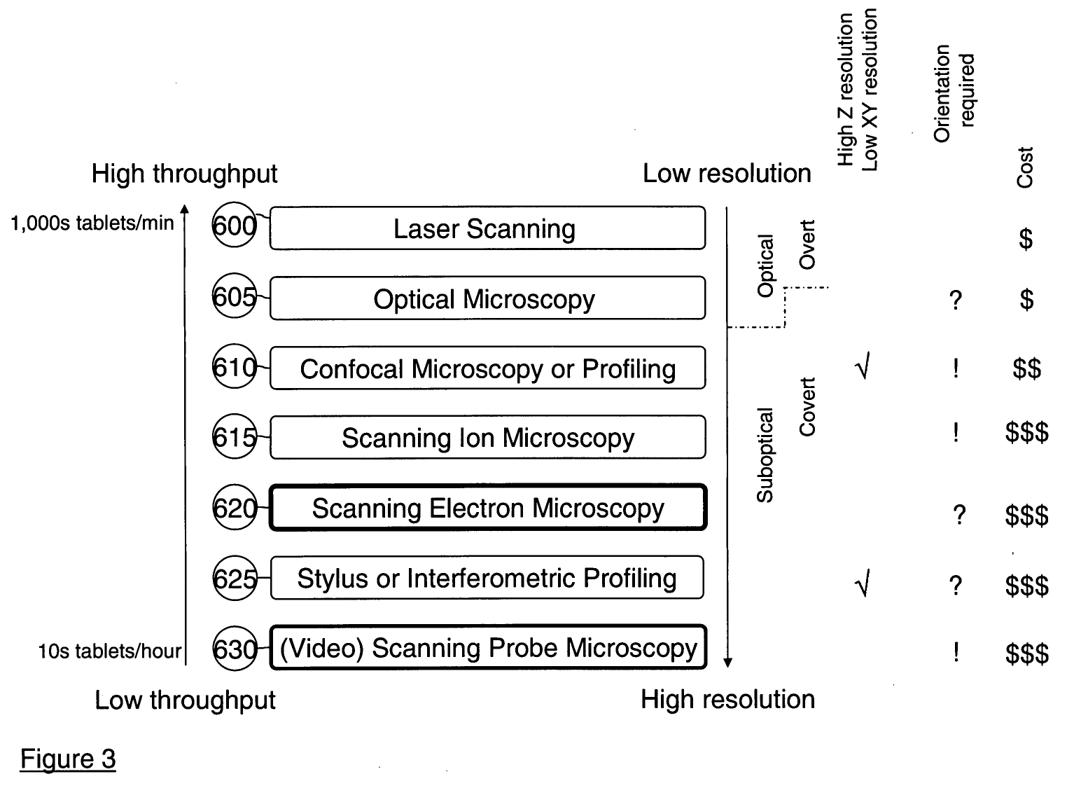

In the following, different embodiments for the inspection step or imaging step (1-230) are given. There are many different techniques that can be used for inspection, each of them with its own advantages and disadvantages. Generally, there is a trade-off (see FIG. 3) between cost and resolution as well as between resolution and throughput. On the side of high throughput with low resolution and low cost are various optical techniques. Laser Scanning (3-600), Optical Microscopy (3-605) and Confocal Microscopy (3-610) are in this group. Next are particle scanning devices such as Scanning Ion Microscopy (3-615) and Scanning Electron Microscopy (3-620). Various Scanning Probe Microscopies (3-630) are the low throughput end of the scale. Stylus or Interferometric Profiling (3-625) as well as confocal microscopy (3-610) produce very high resolution in one direction (Z-resolution) but have low resolution in the orthogonal plane (XY resolution).

### High-Throughput Scanning Probe Microscopy

In another embodiment, provided is a method of decoding the information content of a potentially encoded pharmaceutical composition, the method comprising the steps of: (a) providing a potentially encoded pharmaceutical composition; (b) inserting the composition into a scanning apparatus; (c) reading the surface at high resolution using said scanning apparatus; (d) producing a 2D or 3D image from the acquired data; (e) recognizing the encoded information; (f) decoding the encoded information; (g) presenting the decoded information or comparing it to a database and then reporting the finding. In a preferred embodiment, the scanning apparatus is or derives from a scanning probe microscope.

Also provided is an instrument comprising a scanning probe microscope and a pharmaceutical unit holder holding a pharmaceutical unit. Such a scanning probe microscope preferably should have high throughput which comprises (a) automatic sample and probe handling, (b) optionally automated probe characterization; and (c) fast scan speed.

In most scanning probe microscopies (SPM), a physical small probe is scanned close to the surface of interest and measures a localized physical effect. Thus, SPM produces a map of an area when the probe is scanned in a two-dimensional fashion over the surface. In a preferred embodiment, the technique used is Atomic Force Microscopy (AFM) and the probe is in contact with (contact mode) the surface or very close to it (AC mode) while scanning. During the scan operation, the probe is slowly eroded or damaged either because of friction forces in contact mode or imperfections in the probe positioning system in AC (non-contact) mode. At some point in time, the probe may have to be replaced with a new probe and it can be made sure that the new probe is of high quality. To address automatic probe handling, robotic systems can be used to replace worn probes with high reliability and speed. SPM or assimilated systems such as the Veeco Dimension Vx340 (Santa Barbara, Calif.) and the FEI SNP XT (FEI, Hillsborough, Oreg.) exist that demonstrate the possibility to automatically characterize the probe and set all parameters important for automatic operation.

The sample handling may comprise a tray holding the pharmaceutical units. The tray can be automatically moved using a stage system that is independent of the stage system used for SPM operation. Manufacturers such as Schneeberger (Bedford, Mass.) and Aerotech (Pittsburgh, Pa.) offer many solutions to move trays for millimeter to meter distances, while keeping the location steady within a few nanometers once a location is reached.

An SPM system for high-speed inspection would then accept a tray with units, move the first unit to the probe, approach the probe to the unit, scan the unit, retract the probe from the unit and move the second unit to the probe, etc. in a step and repeat fashion.

Typical SPM systems scan the probe at a rate of about 1-3 scan lines per second. For an image with 256 lines, this means that image acquisition takes 85 s to 256 s. For higher throughput, faster scan speed is important. In one embodiment, Veeco (Santa Barbara, Calif.) Nanodevices Active AFM probes are used. These devices incorporate a piezo actuator in the cantilever and can react faster to changing topography. Consequently, they allow for up to 10× faster scans while still tracking the surface with high fidelity.

In another embodiment, a newly designed video-rate scanning system is used, developed by Infinitesima Limited (Bristol, UK). Here, the scanning stage typically doesn't consist of discrete motor components that can be run at a variety of speeds. Instead, a resonant setup is used that allows probe movement only at the speed corresponding to the resonant frequency of the stage setup which is typically much higher than the highest speed of traditional stage systems. This fast stage is coupled with an active resonance controller that allows for much faster reaction to changes in feedback signal, resulting in imaging speeds up to video rate, that is 1/50 s per image acquisition.

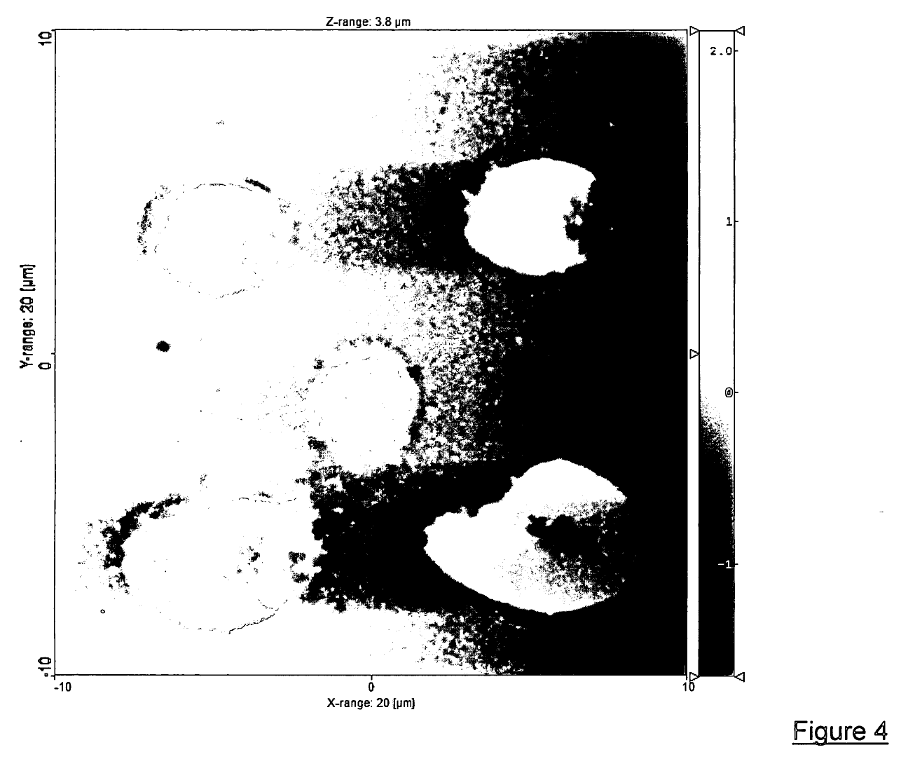

Another technique to improve imaging throughput is to measure a different physical effect and use different feedback parameter in a conventional AFM. For example, using an SIS Picostation system (Herzogenrath, Germany) and turning off the feedback system, one can observe the actual cantilever deflection as the attached probe scans over the surface, rather than keeping the cantilever at the same state of bending during the entire scan as is usual for feedback operation. FIG. 4 shows this so-called error signal for a scan over 5 raised dot microstructures at a speed of 13.25 lines/second. The surface topography can be very well deduced from this very fast scan.

Another technique to improve imaging throughput with SPM is to reduce the high tablet-to-tablet latency time. The probe can first approach the surface and establish feedback before scanning can commence. This approach time is typically on the order of at least 10 seconds. To reduce this time, an accurate knowledge of the sample surface location can be used to move the probe very close without feedback control. This can be accomplished by designing the tablet tray in a way that all tablets extend the same distance from a reference location and by leveling the tablets well. In addition, encoded motors can be used to precisely move the probe close to the surface. In addition, an optical interferometer or laser beam system can find the surface of the tablet precisely and supply the distance the probe needs to move to be very close to the surface.

The following literature related to high-throughput AFM imaging are hereby incorporated by reference in their entirety and can be used to practice the high-speed AFM decoding embodiments:
1. U.S. Pat. No. 5,144,833 published Sep. 8, 1992 to Amer and Meyer (“Atomic Force Microscopy”).
2. U.S. Pat. No. 5,345,815 published Sep. 13, 1994 to Albrecht et al. (“Atomic Force Microscope Having Cantilever With Piezoresistive Deflection Sensor”).
3. Japanese Patent 7,244,054 A2 published Sep. 19, 1995 to Sakata et al. (“Scanning Probe Microscope Device”).
4. U.S. Pat. No. 5,463,897 published Nov. 7, 1995 to Prater et al. (“Scanning Stylus Atomic Force Microscope With Cantilever Tracking And Optical Access”).
5. U.S. Pat. No. 5,595,942 published Jan. 21, 1997 to Albrecht et al. (“Method Of Fabricating Cantilever For Atomic Force Microscope Having Piezoresistive Deflection Detector”).
6. U.S. Pat. No. 5,666,190 published Sep. 9, 1997 to Quate and Minne (“Method Of Performing Lithography Using Cantilever Array”).
7. U.S. Pat. No. 5,675,154 published Oct. 7, 1997 to Lindsay and Jing (“Scanning Probe Microscope”).
8. U.S. Pat. No. 5,742,377 published Apr. 21, 1998 to Minne, Quate and Manalis (“Cantilever For Scanning Probe Microscope Including Piezoelectric Element And Method Of Using The Same”).
9. U.S. Pat. No. 5,760,300 published Jun. 2, 1998 to Kajimura (“Scanning Probe Microscope”).
10. U.S. Pat. No. 5,804,710 published Sep. 8, 1998 to Mamin, Rugar and Terris (“Atomic Force Microscope System with Multi-Directional Voice Coil Actuator for Controlling the Stylus”).
11. U.S. Pat. No. 5,834,644 published Nov. 10, 1998 to Shao et al. (“Automatic Atomic Force Microscope With Piezotube Scanner”).
12. U.S. Pat. No. 5,866,805 published Feb. 2, 1999 to Han et al. (“Cantilevers for a Magnetically Driven Atomic Force Microscope”).
13. U.S. Pat. No. 5,883,705 published Mar. 16, 1999 to Minne et al. (“Atomic Force Microscope For High Speed Imaging Including Integral Actuator And Sensor”).
14. U.S. Pat. No. 6,138,503 published Oct. 31, 2000 to Ray (“Scanning Probe Microscope System Including Removable Probe Sensor Assembly”).
15. PCT Application WO0067290 A2 published Nov. 9, 2000 to Murray, Kim, Chang (“Integrated Microcolumn And Scanning Probe Microscope Arrays”).
16. PCT Application WO0067290 A3 published Nov. 9, 2000 to Murray et al. (“Integrated Microcolumn And Scanning Probe Microscope Arrays”).
17. U.S. Pat. No. 6,310,342 published Oct. 30, 2001 to Braunstein et al. (“Optical Microscope Stage For Scanning Probe Microscope”).
18. U.S. Pat. No. 6,369,385 published Apr. 9, 2002 to Murray et al. (“Integrated Microcolumn And Scanning Probe Microscope Arrays”).
19. U.S. Pat. No. 6,422,069 published Jul. 23, 2002 to Shimuzu et al. (“Self-Exciting and Self-Detecting Probe and Scanning Probe Apparatus”).
20. PCT Application WO02080187 A1 published Oct. 10, 2002 to Hagleitner et al. (“Array and Method for Quasi-Parallel Probe Microscopy”).
21. U.S. Pat. No. 6,469,293 published Oct. 22, 2002 to Shimuzu et al. (“Multiprobe and Scanning Probe Microscope”).
22. U.S. Pat. No. 6,545,492 published Apr. 8, 2003 to Altmann and Horber (“Multiple Local Probe Measuring. Device and Method”).
23. U.S. Pat. No. 6,583,411 published Jun. 24, 2003 to Altmann and Horber (“Multiple Local Probe Measuring Device and Method”).
24. U.S. Pat. No. 6,608,307 published Aug. 19, 2003 to Baur (“System and Method for Accurate Positioning of a Scanning Probe Microscope”).
25. U.S. Pat. No. 6,798,226 published Sep. 28, 2004 to Altmann et al. (“Multiple Local Probe Measuring Device and Method”).

### Scanning Electron Microscopy

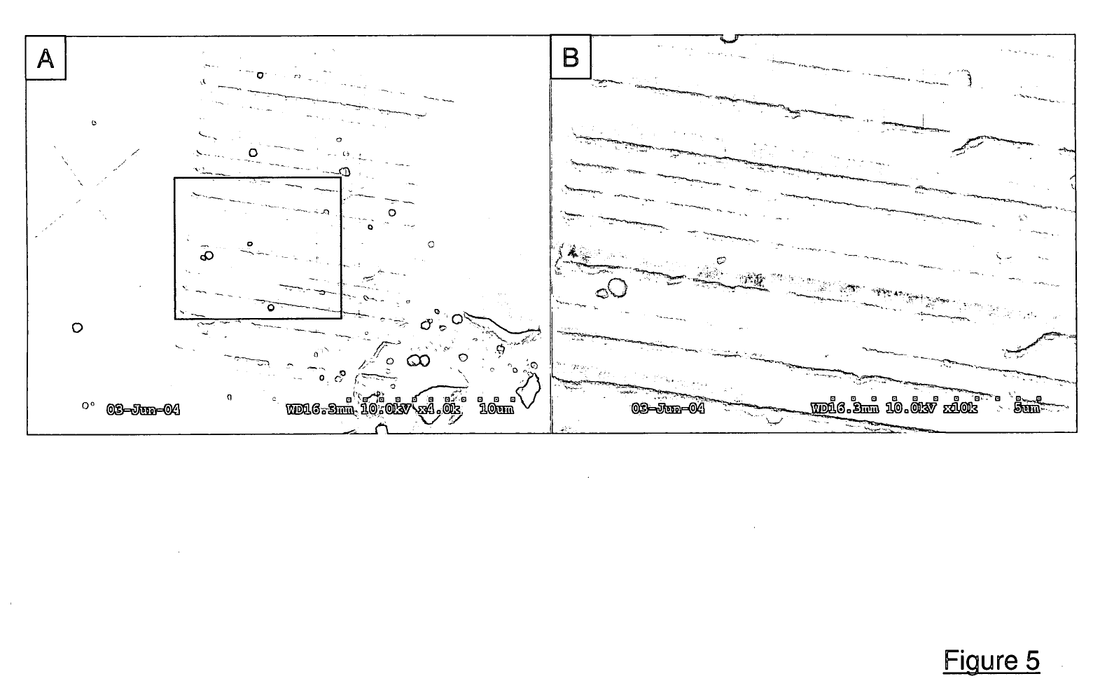

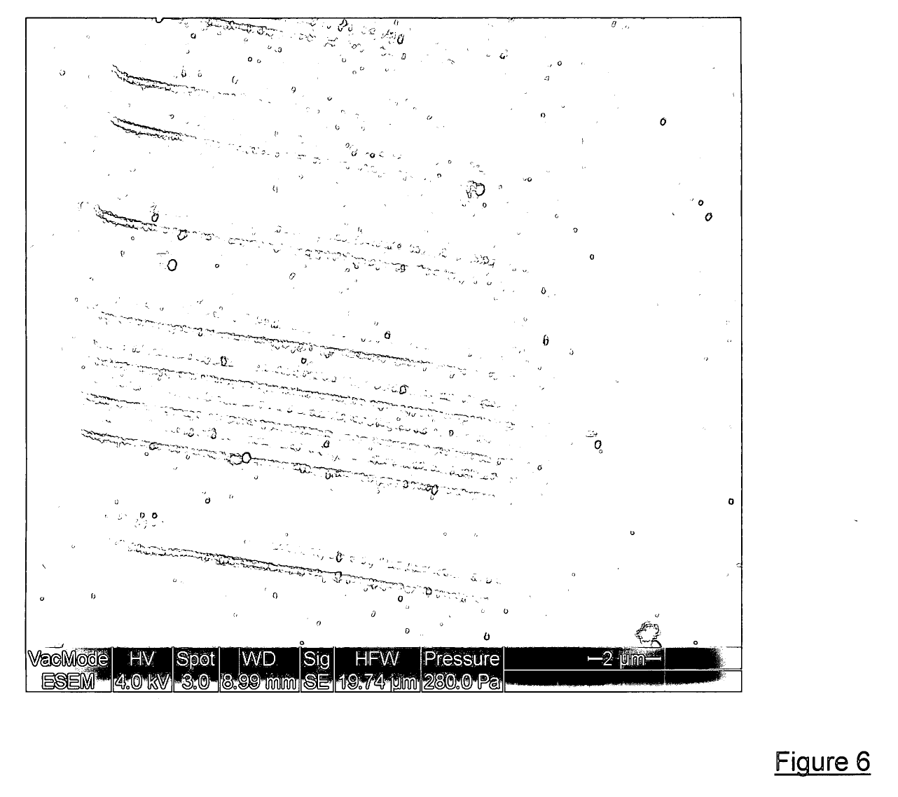

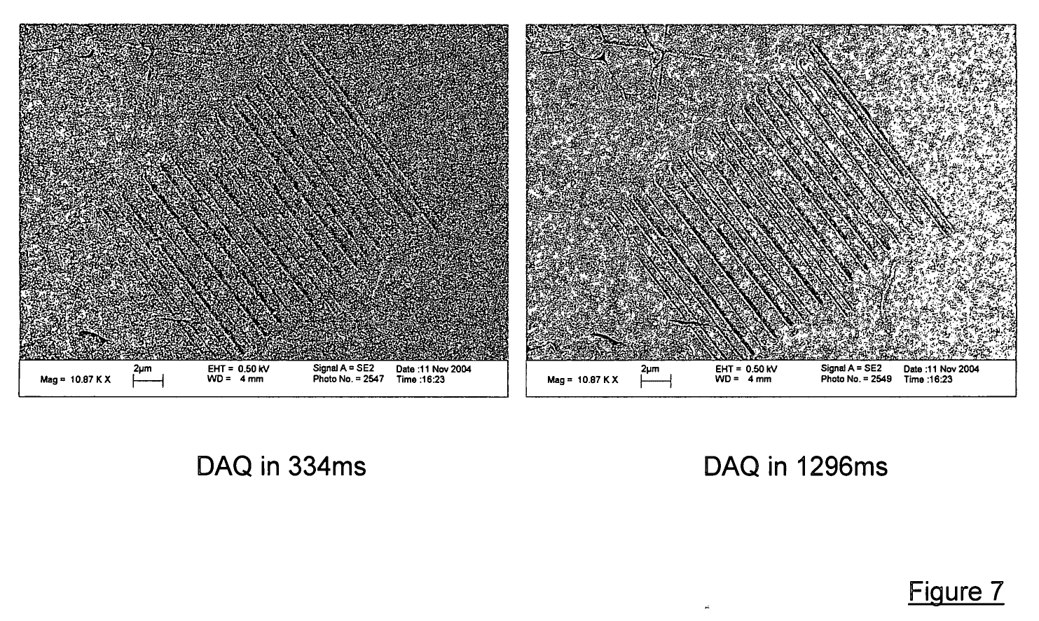

In this preferred embodiment, provided is the method of determining if a pharmaceutical composition is genuine or fake using a scanning electron microscope, the method comprising the step of examining an object or composition potentially bearing at least one information-encoding feature with said scanning electron microscope (SEM). Scanning Electron Microscopes (SEMs) are well suited to high-speed inspection of tablets with information-bearing marks because they produce contrast on the edges and topography changes of the mark and can quickly acquire data. Their staging system is well suited to automation and the introduction of a tray that contains many tablets. SEM imaging on pharmaceutical units was performed with metal-coated tablets using a Hitachi S-3000N instrument with a tungsten electron source and 20 kV acceleration voltage (see FIG. 5), with an FEI Quanta series 200 system using environmental SEM mode at 280 Pa pressure and 4 kV acceleration voltage (see FIG. 6) and a LEO-Zeiss (Jena, Germany) 1525 FEG SEM with a Schottky diode electron source at 0.5 kV, high vacuum and no modification to the tablet (see e.g. FIG. 7).

Further provided is an automated apparatus based upon a scanning electron microscope for detecting if at least one object or composition is genuine or fake, the apparatus comprising at least one holding member holding at least one object or composition, an imaging mechanism comprising a vacuum or low pressure analysis chamber adapted to contain said holding member, and an electron beam column adapted to generating and scanning an electron beam, as well as at least one electron detector. The apparatus may further comprise a loading system to load said holding member into the apparatus and/or a degassing member wherein the object and composition may be brought from ambient pressure to low pressure or vacuum without busying the analysis chamber. The apparatus may further comprise a computing device running an algorithm that enables locating, imaging and decoding of identification features on said object or composition according to any of the aforementioned methods.

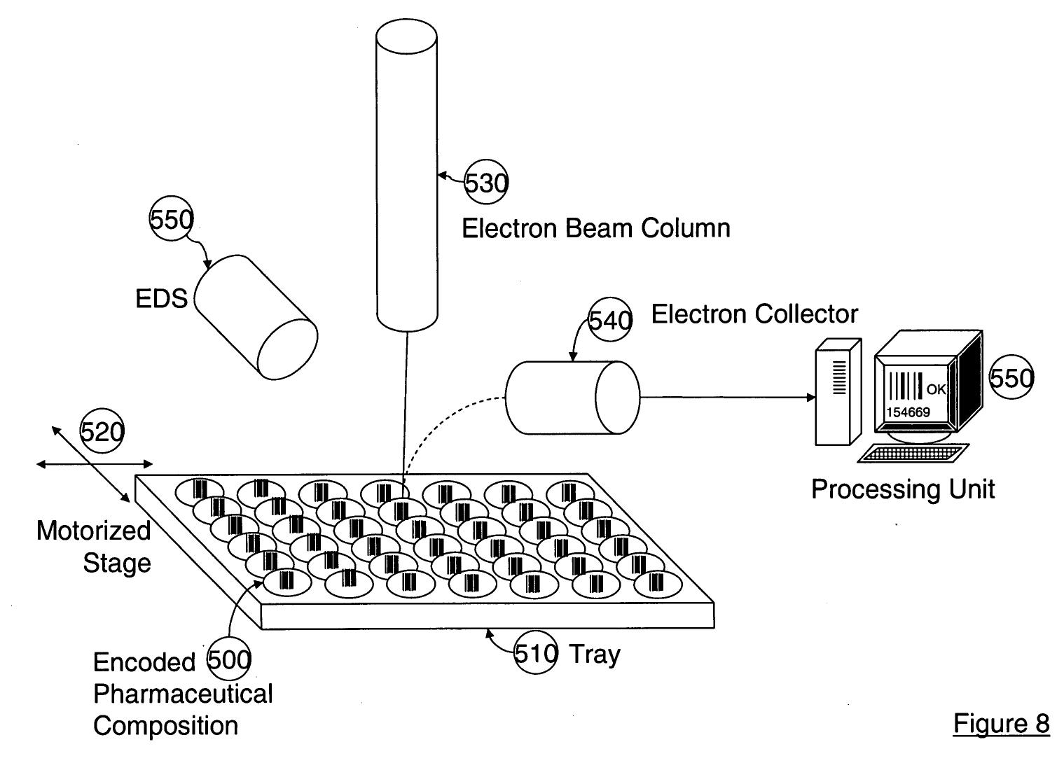

FIG. 8 shows a typical embodiment of such an inspection system using an SEM. A tray (8-510), containing properly oriented and placed pharmaceutical compositions (8-500) and mounted on a motorized stage (8-520) with a least two axes of movement, is placed inside a SEM vacuum chamber. There, the tray is positioned underneath the Electron Beam Column (8-530) which is the source of the probing electrons. The electron beam is scanned over one pharmaceutical unit. Various detectors can be used to detect effects of the electrons scanning over the unit. Preferably, the detector used is an in-lens electron detector that is placed very close to or into the electron beam column (8-530). Preferably, the detector used is a secondary electron detector (8-540) that is placed further away from the column. Optionally, an energy-dispersive X-ray detector (EDS, 8-550) is used to measure secondary effects of the electron bombardment (e.g. provides independent confirmation that an actual tablet is imaged via analysis of the EDS chemical information). The signal from the detector is send to a processing unit (8-550) that relates the electron-beam movement to the incoming data stream and produces a two-dimensional image of the scanned surface. The SEM information can be matched up with the motorized stage location which gives information about the particular unit imaged. The SEM information is saved together with the label of the imaged unit and further processed from there on.

To improve throughput, the tray may not be directly loaded into the vacuum chamber which can involve pump-down time before imaging can commence. Instead, a filled second tray can be placed into a load-lock that is attached to the main SEM vacuum chamber and is evacuated while the first filled tray is processed. Once each unit on the first tray is imaged, the first and second trays are exchanged in the load-lock and imaging of the second tray can commence without further delay. The first tray can be removed and either re-stocked with new unit or replaced with a third tray that will be pumped down by the time the second tray is finished.

### Other Imaging Embodiments

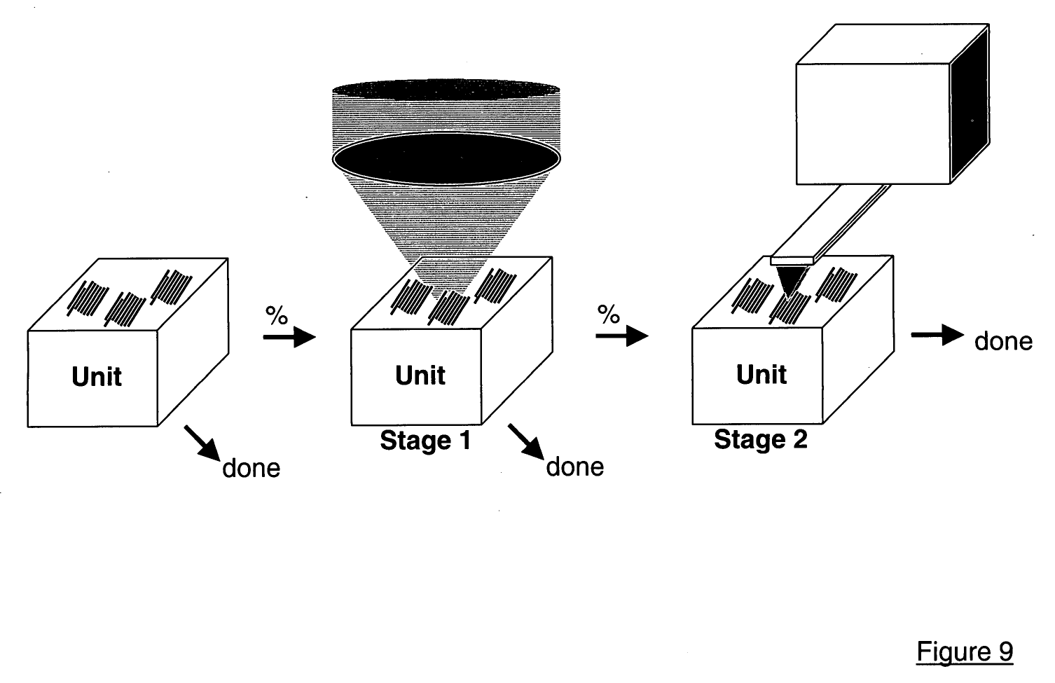

Many other types of inspection exist that can be applied for either readout of the information bearing marks or as a first step to identify existence and location of the information bearing marks. In one embodiment of this strategy (see FIG. 9), for example usable for quality control after producing the marks, the unit would be sampled at a certain percentage of full output with a first inspection system that could for example be of optical nature. Certain larger marks could give good evidence of the completeness of the smaller mark. Thus inspecting these larger marks with the first inspection system can act as a first level of quality control by deciding if the larger marks are in good condition. Furthermore, the first inspection system can locate the larger mark and relate it to the location of the smaller mark such that the second, higher-resolution inspection system has a high probability of finding the smaller mark in a small area given by the result of the inspection with the first inspection tool. A certain percentage of the pharmaceutical units inspected with the first inspection tool can be sampled with the second inspection tool and result in imaging of the smaller information-bearing mark.

Other combination of coarse- and high-resolution imaging system may be used. For example, laser scanning devices, such as those offered by Keyence (Woodcliff Lake, N.J.), are among the fastest and least accurate imaging systems available for this purpose. A laser is scanned over the surface and produces displacement and surface profile measurements, independent of surface angle or color. These systems can for example be used to find the general area where the mark or ensemble thereof is on e.g. the pharmaceutical unit. The laser scanner may also be useful to read a larger information bearing mark on the unit's surface.

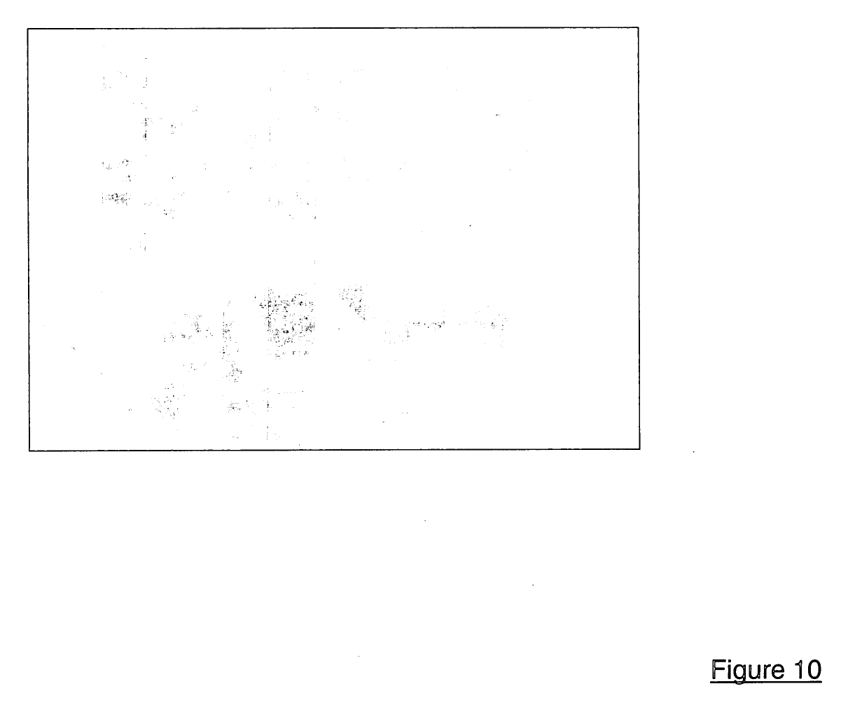

Optical microscopy is another possibility either as a coarse-resolution technique for directly or indirectly locating the micron/nanometer-scale identification features or for direct use for imaging micron-scale identification features if using some of the most advanced methods. General standard optical microscopy can be used to find the general area of the identification feature(s), or find and identify larger information bearing marks with known spatial relationship of the micron- or nanometer-scale information bearing mark as seen in FIG. 10.

Special optical techniques with dedicated high-resolution instruments can be used to decode some of the larger micron-scale information-bearing marks. Such techniques include for example immersion lens technology, UV illumination or metal coating. Immersion lenses can be made with a larger numerical aperture than standard microscope lenses because the refractive index of all media from the lenses to the sample can be controlled. In contrary, standard optical microscopy utilizes an air-gap between the sample and the lens and thus has limited optical engineering options.

The Rayleigh diffraction law states that the diffraction limited resolution limit of a microscope is proportional to the wavelength used. Thus, using UV illumination, more resolution can be gained than for visible white light or visible monochromatic light of any color.

Metal-coating of pharmaceutical tablets enhances optical imaging and allows resolution of features that would, given the transparency and scattering of some pharmaceutical tablets

Confocal microscopy may also be used. In confocal microscopy, the microscope's condenser and objective lenses both focus onto one single point (or a single plane) of the sample. Generally, the image of a pinhole source is focused onto a point on the sample, and that point is focused by the objective lens onto a point detector or through a mask with a pinhole aperture. With confocal optics, the Rayleigh limit of resolution may be exceeded since only a limited region of the sample is viewed at any one time. Confocal microscopy's high Z-resolution may also be utilized to sense the depth of the identification features as depth can be used to serve as an identification feature. This can be important when, for example, at least one identification feature can be imprinted into the target object or composition and the corresponding identification information can be encoded in the depth of the different components of said identification feature(s).

Scanning ion microscopes (SIM) are charged-particle imaging systems operating similarly to scanning electron microscopes. Among Scanning ion microscopes, the focused ion beam (FIB) system using Gallium ions is the most developed type. For detection, either secondary electrons or secondary ions can be used. SIM can be used as an imaging member in pharmaceutical tablet authentication.

Stylus or interferometric profiling machines can also be used as an inspecting member in pharmaceutical tablet authentication. They can either be powered by a stylus not unlike the probe of a scanning probe microscope, or they can use interference of light. Both have in common extremely good resolution in z-direction, normal to the surface within single nanometers or less. Both also have in common relatively poor resolution in x- and y-direction, parallel to the surface of typically a few micrometers. Thus very closely spaced features such as those incorporated in small information bearing marks may not be imaged with high resolution using a profiling instrument. However, profilometers can be used to find the general area of identification features on an object or composition or read larger scale information bearing mark, esp. these that would be encoded by depth.

A typical optical profilometer using interferometric distance detection is the Veeco (Santa Barbara, Calif.) Wyko NT1100 Optical Profiler. It produces sub-nm vertical resolution. However, for optical profilometers, the surface to be measured may have to have certain optical properties, such as a minimum reflectance and a certain maximum angle versus normal for very shiny surfaces. A typical stylus profilometer is the Veeco (Santa Barbara, Calif.) Dektak 6M Bench-Top Stylus Profilometer. It uses a feedback-less sharp stylus that is dragged over the surface. This stylus senses the surface. A one-dimensional or two-dimensional scanning pattern is used to step height or surface height data.

### Data Extraction and Processing

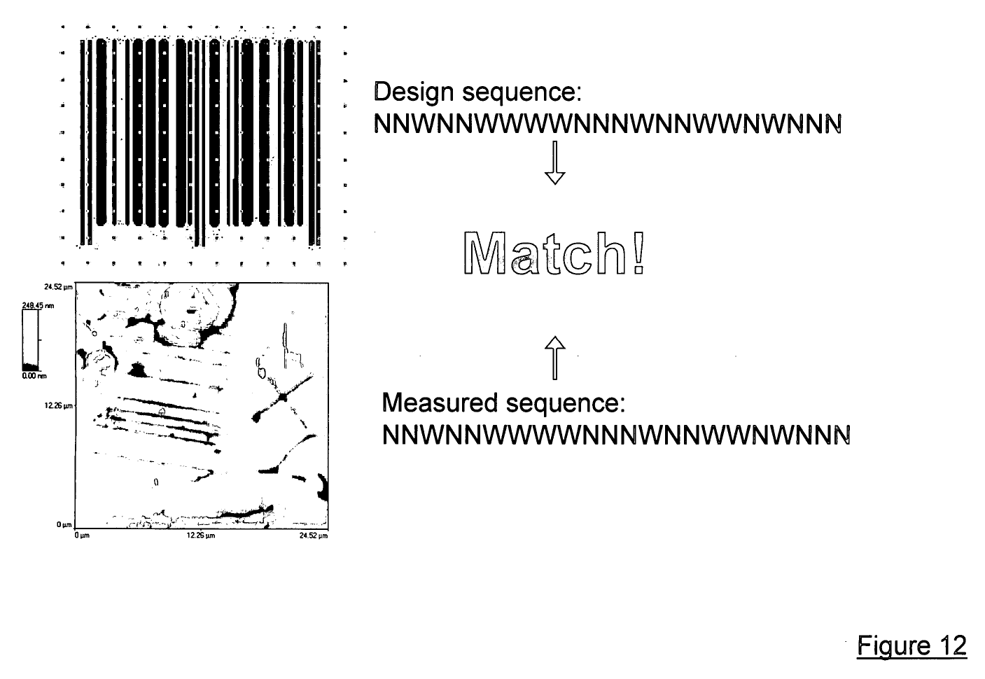

FIG. 12 illustrates the steps of locating and imaging an identification feature, measuring some of its characteristics (e.g. the width of individual lines in the pattern, N=narrow, W=wide) to extract identification information (in this case, a binary string equivalent to a number or alphanumeric string) and comparing said information with a stored value in a database in order to identify the item under inspection as genuine or not. Other characteristics of the identification features may be measured:
Distance between marks,
Locations,
Type of overt/covert mark,
   There may be a mathematical algorithm that combines one or more geometric characteristics (along all axis e.g. X, Y, Z) or others to generate the identification information.

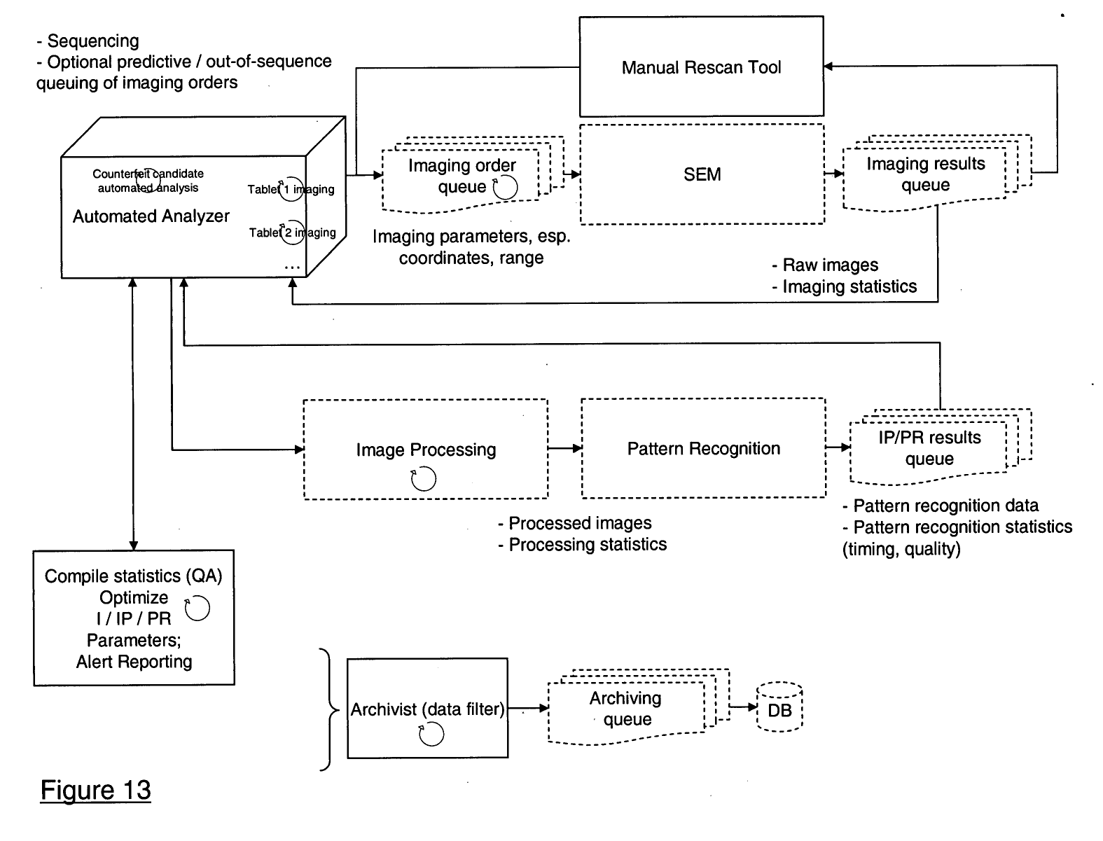

FIG. 13 illustrates the different components of a pharmaceutical tablet inspection instrument based upon a Scanning Electron Microscope (SEM) that can determine the presence or absence of substantially covert, micro- and nanoscale identification features printed or imprinted on said objects and compositions, decode the information stored in said identification features, if any; and utilize said determination and said information to identify said objects or compositions as genuine, fake or illegally traded. In FIG. 13, IP is image processing; PR is pattern recognition; DB is database.

Again, the database can also be a plurality of databases including a database provided by a company doing the authentication as well as a pharmaceutical company.

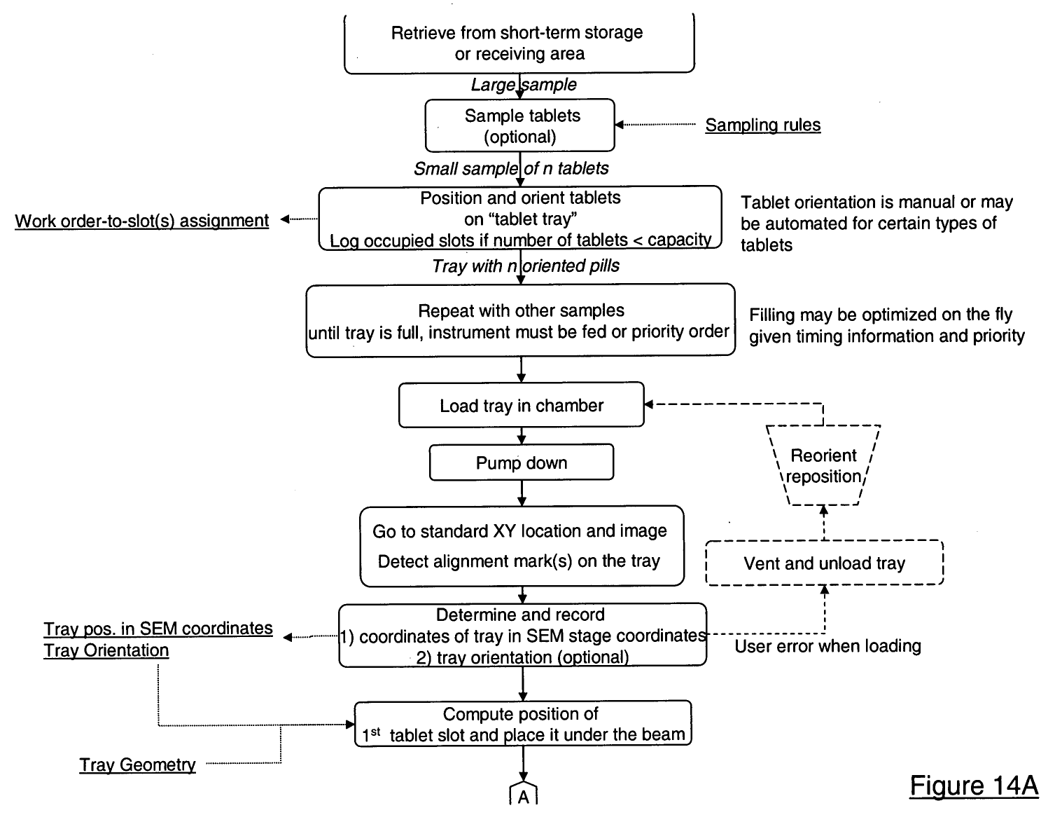

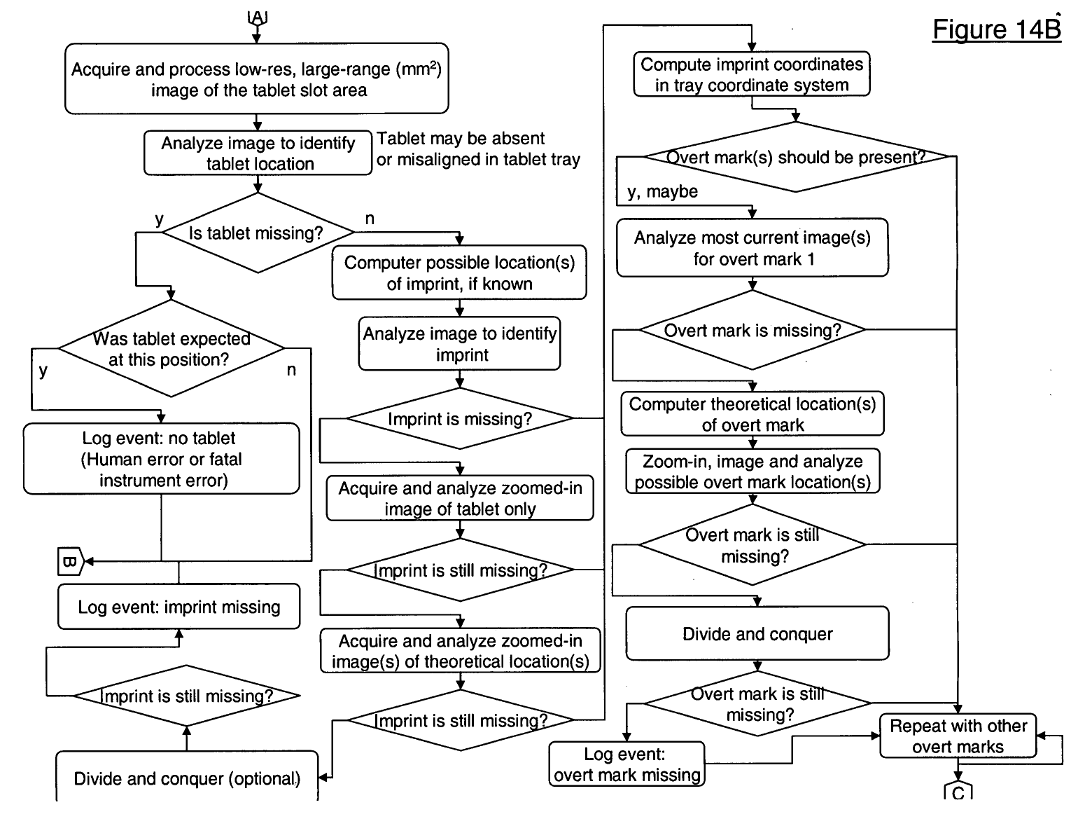

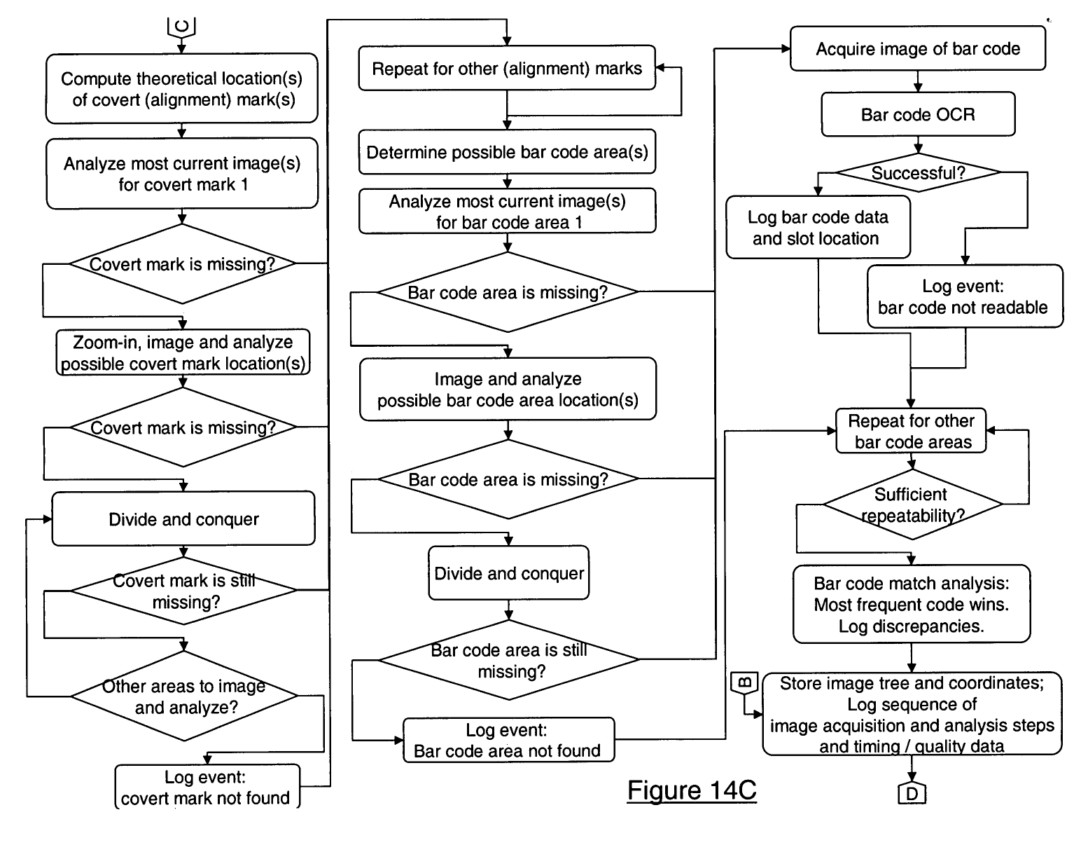

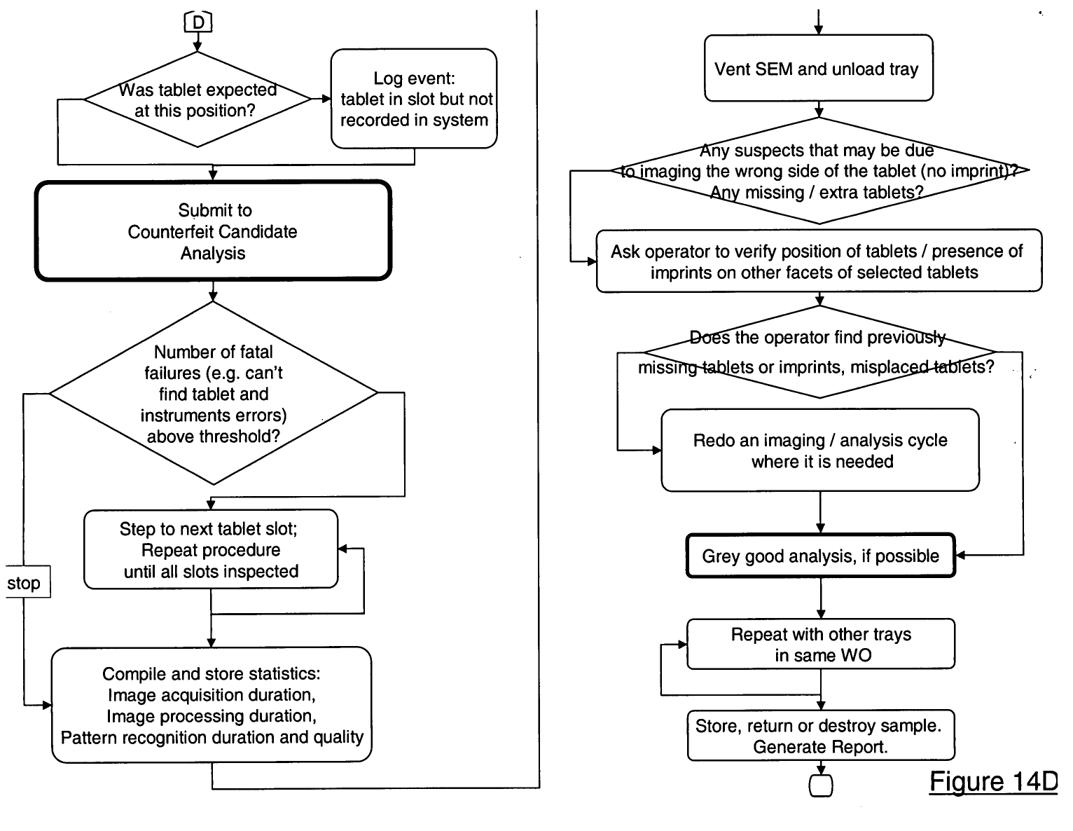

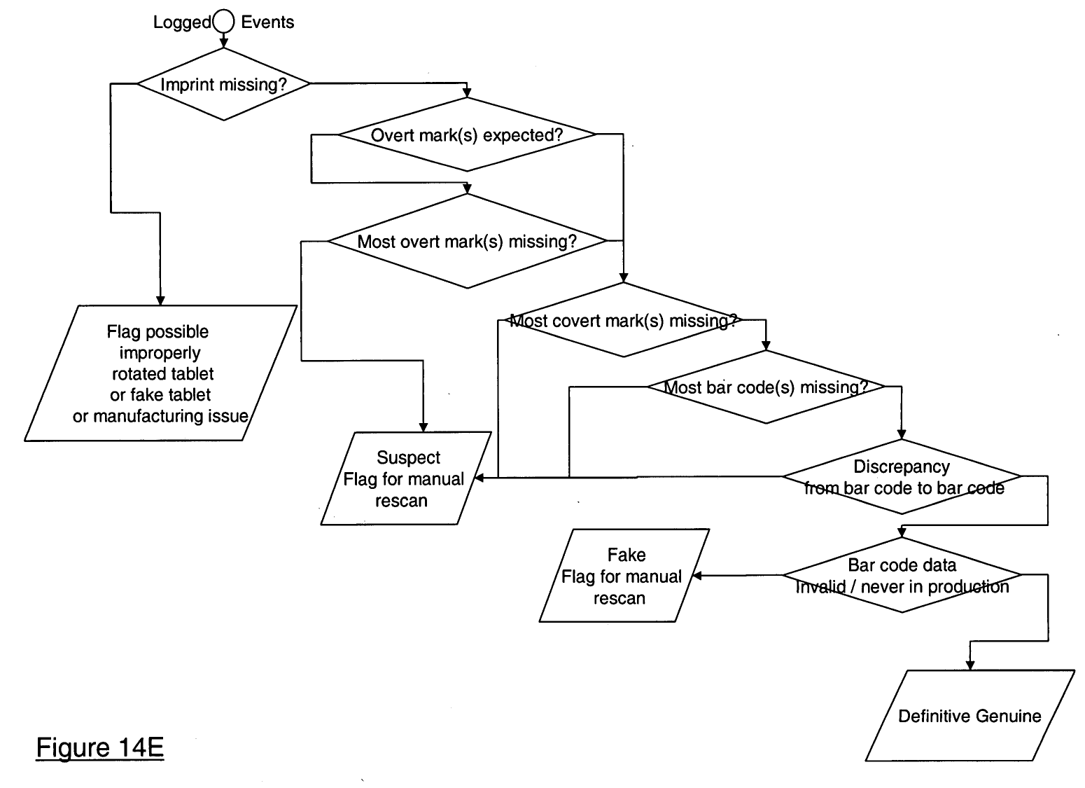

FIGS. 14A, B, C, D and E illustrate a detailed, preferred algorithm to be run by the computing device part of the pharmaceutical tablet inspection instrument shown in FIG. 13. The algorithm includes pattern extraction, pattern recognition (or OCR), data decoding and database lookup steps that are known to the art of computer engineering and image processing. The algorithm provides the following advantages:
Minimization the number of image acquisition steps,
Minimization of the number of image analysis steps,
Adaptation to variations during identification feature fabrication (including various generations of technologies),
Analysis in depth to avoid false positives due to human errors,
Partial counterfeits detection,
Counterfeiting checks at multiple levels,
Parallelism between imaging, image processing/pattern recognition, counterfeit analysis and other background tasks
Maximization of SEM and pattern recognition duty cycle (imaging queue, image processing queue)

In other embodiments related to FIG. 14 including FIGS. 14D and 14E, storage of units can be carried out for use as evidence in criminal proceedings and prosecution. Drugs can be separated and checked individually. Again, the data bases can include data bases from the company doing authentication as well as data bases from a pharmaceutical company.

### Other Objects

As described above, a particular preferred example of the invention is a unit pharmaceutical composition, and methods of identifying the unit pharmaceutical composition. In general, the various embodiments of the invention can be applied to pharmaceutical goods which are susceptible to counterfeiting, including for example high priced pharmaceuticals, prescription drugs, and blockbuster drugs with large sales volume, wherein price differentials exist from country to country and the economic incentive to counterfeit is high, as described above. The description above for pharmaceutical compositions can be also adapted to apply to other compositions and objects, in unit form for example, which can be subjected to counterfeiting fraud such as the confectionary compositions and consumer goods like CDs or DVDs, as well as medical devices, such as stents and catheters, and similar supplies. Additional preferred examples of objects include currency, consumer products, paper, money, documents, entertainment media, compact disks, DVDs, nickel masters, flat wafers, disk drive heads, semiconductor chips, integrated circuits and their components, packaging containers and materials including packaging containers and materials for pharmaceuticals, jewelry, precious raw materials, personal and institutional identification devices, medical devices, bottle tampering-evident seals, syringes, jewelry and collectibles. In particular, syringes, pre-loaded syringes, vaccines and vaccine vials, and injectable drug vials, including bottle seal, medical devices including catheters and implantable devices, and packaging labels can be used. In general, objects which are susceptible to counterfeiting or copying are particularly of use.

### Further Literature

The following patents and co-pending applications related to Dip Pen Nanolithography printing are hereby incorporated by reference in their entirety and can be used to prepare identification features on surfaces of unit pharmaceutical compositions, either directly or indirectly through use of for example stamps and imprinting:
1. U.S. Provisional Application 60/115,133 filed Jan. 7, 1999 (“Dip Pen Nanolithography”) to Mirkin et al.
2. U.S. Provisional Application 60/157,633 filed Oct. 4, 1999 to Mirkin et al. (“Methods Utilizing Scanning Probe Microscope Tips and Products Therefor or Produced Thereby”)
3. U.S. Regular patent application Ser. No. 09/477,997 filed Jan. 5, 2000 (“Methods Utilizing Scanning Probe Microscope Tips and Products Therefor or Produced Thereby”) to Mirkin et al.
4. U.S. Provisional Application 60/207,713 filed May 26, 2000 (“Methods Utilizing Scanning Probe Microscope Tips and Products Therefor or Produced Thereby”) to Mirkin et al.
5. U.S. Provisional Application 60/207,711 filed May 26, 2000 (“Methods Utilizing Scanning Probe Microscope Tips and Products Therefor or Produced Thereby”) to Mirkin et al.
6. U.S. regular application Ser. No. 09/866,533 filed May 24, 2001 (“Methods Utilizing Scanning Probe Microscope Tips and Products Therefor or Produced Thereby”) to Mirkin et al.
7. U.S. Patent Publication 2002/0063212 A1, published May 30, 2002 (“Methods Utilizing Scanning Probe Microscope Tips and Products Therefor or Produced Thereby”) to Mirkin et al.
8. U.S. Patent Publication 2002/0122873 A1 published Sep. 5, 2002 (“Nanolithography Methods and Products Produced Therefor and Produced Thereby”).
9. PCT Publication WO 00/41213 A1 published Jul. 13, 2000 based on PCT application no. PCT/US00/00319 filed Jan. 7, 2000 (“Methods Utilizing Scanning Probe Microscope Tips and Products Therefor or Produced Thereby”).
10. PCT Publication WO 01/91855 A1 published Dec. 6, 2001 based on PCT application no. PCT/US01/17067 filed May 25, 2001 (“Methods Utilizing Scanning Probe Microscope Tips and Products Therefor or Produced Thereby”).
11. U.S. Regular patent application Ser. No. 10/307,515 filed Dec. 2, 2002 to Mirkin et al. (“Direct-Write Nanolithographic Deposition of Nucleic Acids from Nanoscopic Tips”).
12. U.S. Regular patent application Ser. No. 10/320,721 filed Dec. 17, 2002 (“Patterning of Solid State Features by Direct-Write Nanolithographic Printing”) to Mirkin et al.
13. U.S. Patent Publication 2003/0022470 A1, published Jan. 30, 2003 (“Parallel, Individually Addressable Probes for Nanolithography”) to Liu et al.
14. U.S. Patent Publication 2003/0007242, published Jan. 9, 2003 to Schwartz (“Enhanced Scanning Probe Microscope and Nanolithographic Methods Using Same”).
15. U.S. Patent Publication 2003/0005755 to Schwartz, published Jan. 9, 2003 (“Enhanced Scanning Probe Microscope”).
16. U.S. Regular patent application Ser. No. 10/366,717 to Eby et al., filed Feb. 14, 2003 (“Methods and Apparatus for Aligning Patterns on a Substrate”).
17. U.S. Regular patent application Ser. No. 10/375,060 to Cruchon-Dupeyrat et al., filed Feb. 28, 2003 (“Nanolithographic Calibration Methods”).
18. U.S. Patent Publication 2003/049381 A1 to Mirkin et al., published Mar. 13, 2003 (“Methods Utilizing Scanning Probe Microscope Tips and Products Therefor or Produced Thereby”).
19. U.S. Patent Publication 2003/0068446 A1, published Apr. 10, 2003 to Mirkin et al. (“Protein and Peptide Nanoarrays”)
20. U.S. Patent Publication 2003/157254 A1, published Aug. 21, 2003 to Mirkin et al. (“Methods Utilizing Scanning Probe Microscope Tips and Products Therefor or Produced Thereby”).
21. U.S. Patent Publication 2003/162004 A1, published Aug. 28, 2003 to Mirkin, Dravid, Su, Liu (“Patterning of Solid State Features by Direct-Write Nanolithographic Printing”).
22. U.S. Pat. No. 6,635,311 issued Oct. 21, 2003 to Mirkin et al. (“Methods Utilizing Scanning Probe Microscope Tips and Products Therefor or Produced Thereby”).
23. U.S. Pat. No. 6,642,129 issued Nov. 14, 2003 to Liu et al. (“Parallel, Individually Addressable Probes for Nanolithography”).
24. U.S. Pat. No. 6,674,074 issued Jan. 6, 2004 to Schwartz (“Enhanced Scanning Probe Microscope”).
25. U.S. Patent Publication 2004/008330 A1 published Jan. 15, 2004 to Mirkin, Lim (“Electrostatically Driven Lithography”).
26. U.S. Patent Publication 2004/028814 A1, published Feb. 12, 2004 to Mirkin et al. (“Methods Utilizing Scanning Probe Microscope Tips and Products Therefor or Produced Thereby”).
27. U.S. Patent Publication 2004/037959 A1, published Feb. 26, 2004 to Mirkin et al. (“Methods Utilizing Scanning Probe Microscope Tips and Products Therefor or Produced Thereby”).
28. U.S. Pat. No. 6,737,646 issued to Schwartz (“Enhanced Scanning Probe Microscope and Nanolithographic Methods Using Same”).
29. U.S. Patent Publication 2004/119490 A1, published Jun. 24, 2004 to Liu et al. (“Parallel, Individually Addressable Probes for Nanolithography”).
30. U.S. Patent Publication 2004/131843 A1, published Jul. 8, 2004 (“Nanolithography Methods and Products Produced Therefor and Produced Thereby”).
31. U.S. Patent Publication 2004/142106 A1, published Jul. 22, 2004 (“Patterning Magnetic Nanostructures”).
32. U.S. Patent Publication 2004/175631 A1, published Sep. 9, 2004 (“Nanometer-scale engineered structures, methods and apparatus for fabrication thereof, and applications to mask repair, enhancement, and fabrications”).

### WORKING EXAMPLES

### Example 1

### SEM Imaging of Hot-Embossed Pharmaceutical Tablets

FIG. 5[A] is a Scanning Electron Microscopy (SEM) image of an information-bearing mark on a commercial tablet. The tablets were imprinted for 3 s at 130° C. and a force of approx. 80 N and a pressure of approx. 30 MPa. They were then coated with a 3 nm thick film of a Pt/Pd alloy (using a Polaron E5100 sputter coater), grounded with conductive tape to prevent charging and imaged under high vacuum at 10 kV acceleration voltage. A Hitachi S-3000N instrument with a tungsten electron source was used. Imaging in Variable Pressure mode (at a 50 Pa pressure, 20 kV acceleration voltage, without metal coating) was also possible, although with a very low contrast. FIG. 8[B] is a close-up of image [A].

### Example 2

### AFM Imaging of Hot-Embossed Pharmaceutical Tablets

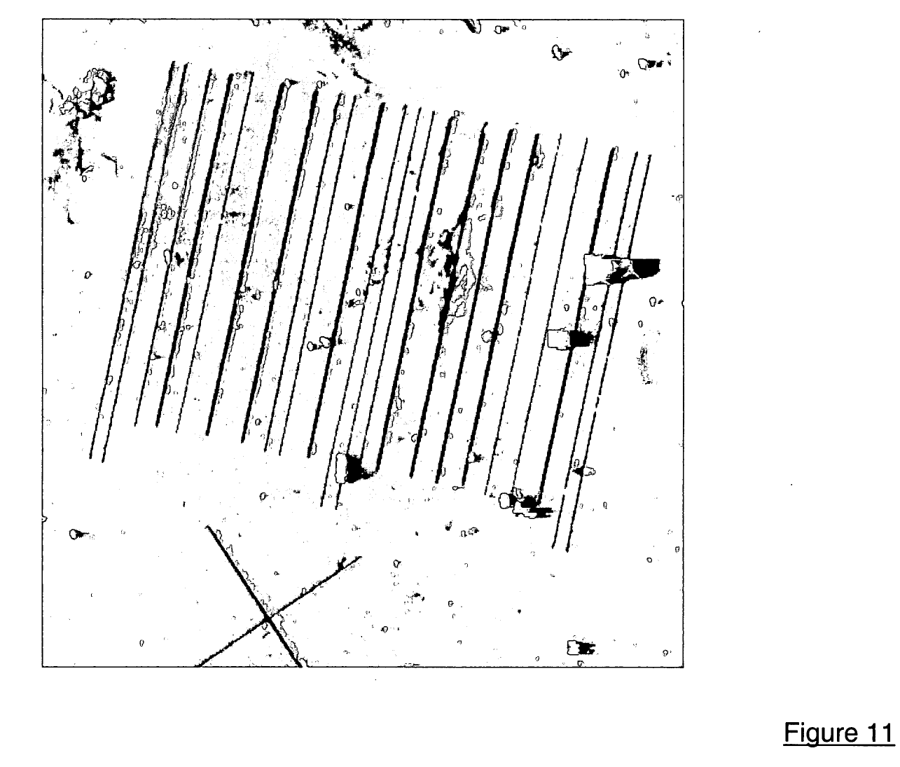

FIG. 11 shows an AFM image of an information-bearing mark on a commercial tablet prepared by NanoInk, Inc. An NScriptor system (NanoInk, Chicago, Ill.) was then used in AFM mode to image the surface of the tablet. A NanoInk contact silicon-nitride probe was brought into feedback with the tablet. The feedback parameters (i.e. the proportional, integral and derivative gain of the feedback loop) were equal to 4, 3 and 0, respectively. These values provide only weak following of the topography. Instead of reading out the topography signal, the error signal was read and is displayed here. The error signal measures the deflection of the cantilever.

### Example 3

### High-Throughput Reading of Encoded Pharmaceutical Tablets

FIG. 10 shows two SEM micrographs of an information-bearing mark on a commercial tablet and produced as described in Example 2. A LEO 1525 FEG SEM with a Schottky diode electron source was used. The tablet was not coated with metal and instead introduced into the scanning chamber with no modifications. Using a low acceleration voltage of 0.5 kV resulted in imaging without charging effects. The image on the left side of FIG. 10 was acquired in 0.33 seconds; the image on the right was acquired in 1.30 seconds. Both images allow the information-bearing mark to be seen and the code deciphered. This proves that an image acquisition time of is realistic and a high throughput of up to 3,600 pharmaceutical units per hour is possible.

### Example 4

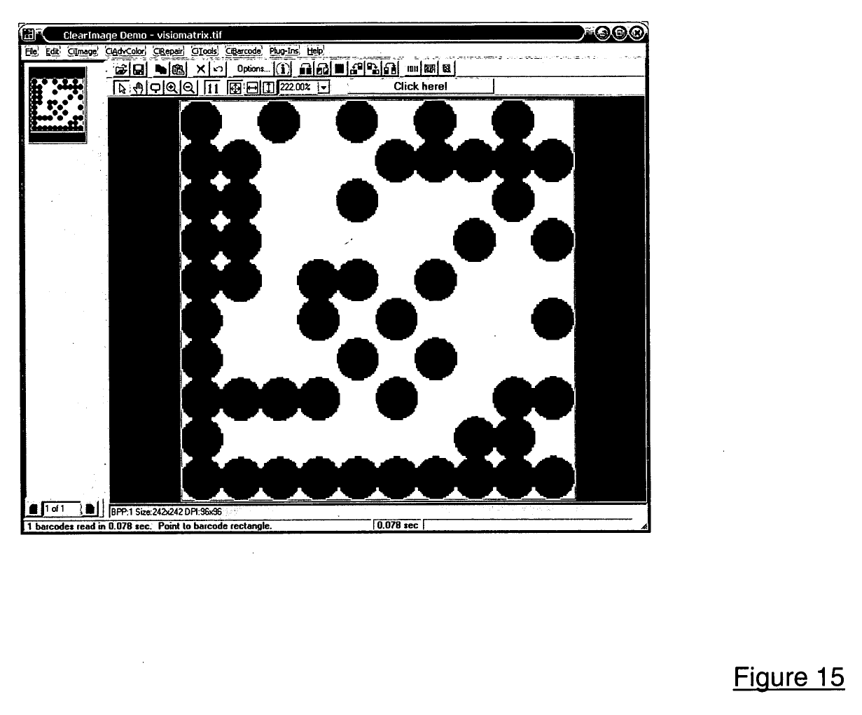

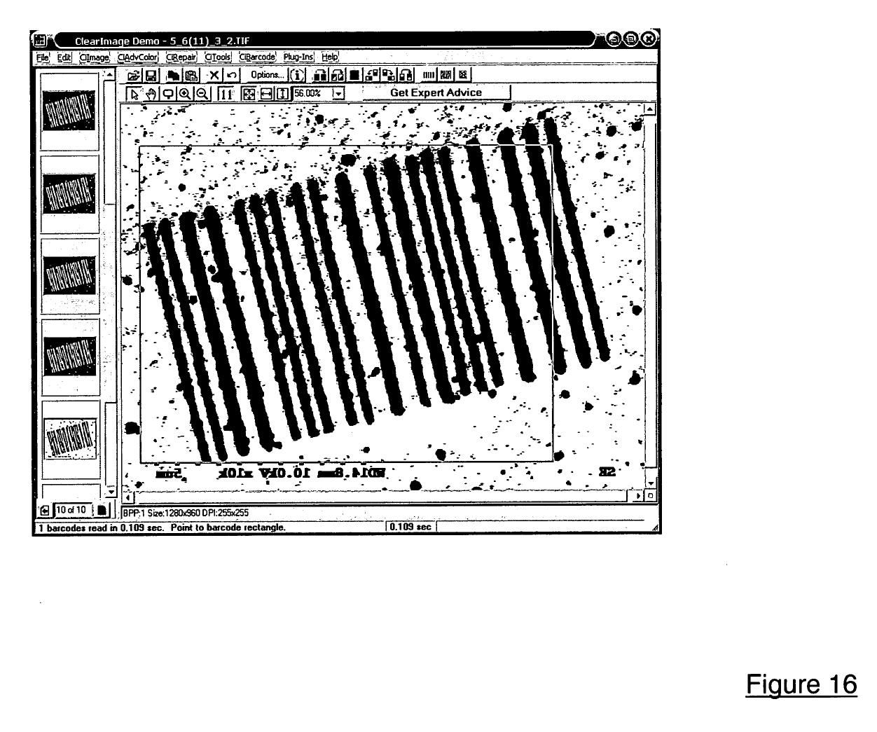

FIGS. 15 and 16 illustrate the decoding of a nanometer-scale Data matrix-style barcode and a linear barcode, respectively. A barcode comprising a set of “dots” positioned at various locations of a grid was processed in a commercial barcode processing software. The string encoded by the position of the dots was successfully decoded from the image that is shown. In FIG. 16, an image of an actual nanometer-scale linear barcode imprinted on a pharmaceutical tablet comprising a set of “lines” of various widths as elements was processed in a commercial barcode processing software. The string encoded by the width sequence in the barcode was successfully decoded from the image that is shown.

### Example 5

FIGS. 17 and 18 provide SEM analysis for imprinted tablets having data matrix imprints.

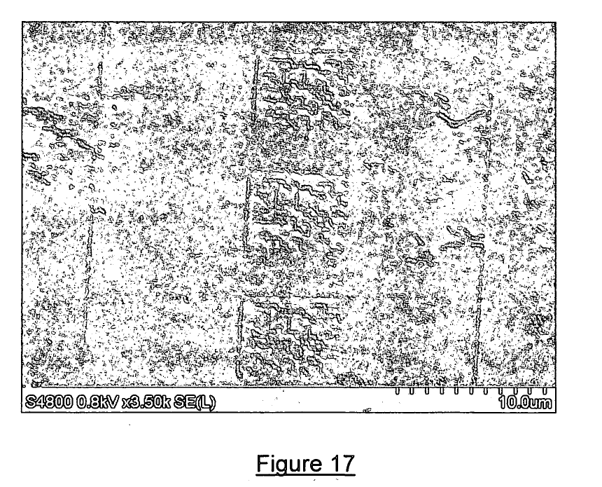

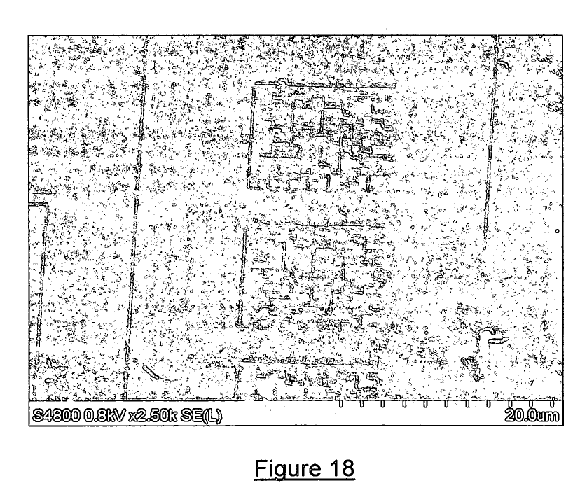

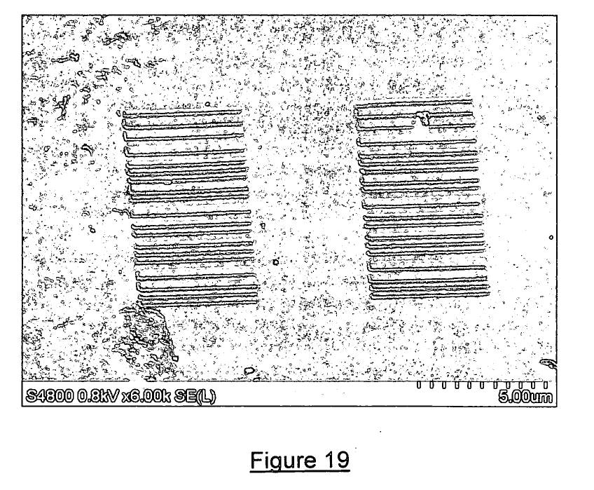

FIGS. 19 and 20 provide SEM analysis for imprinted tablets having barcode imprints.

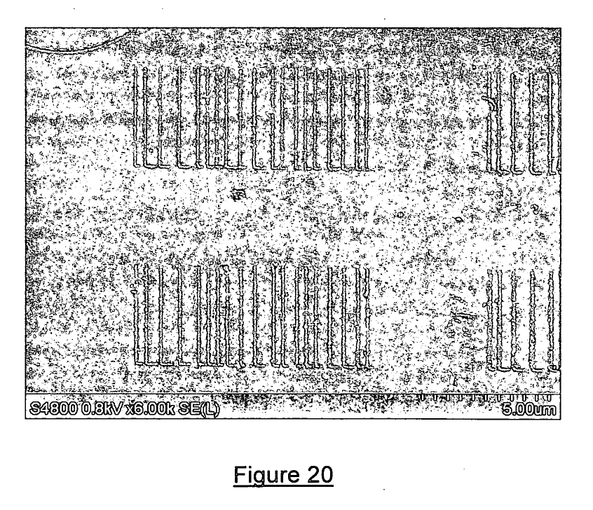

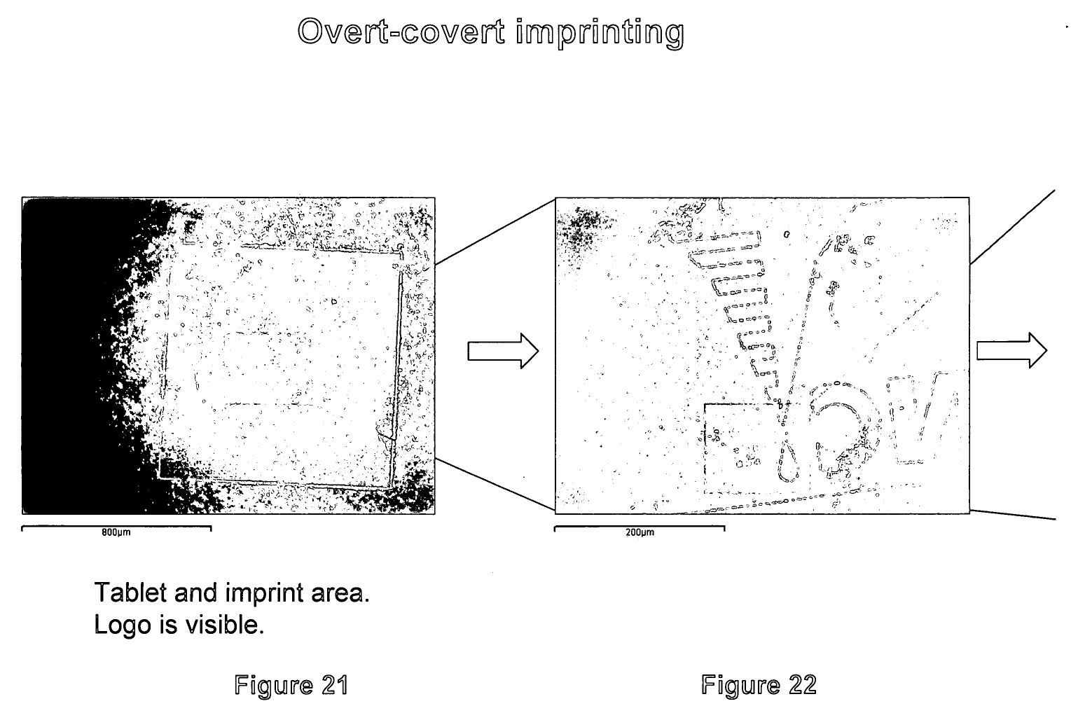

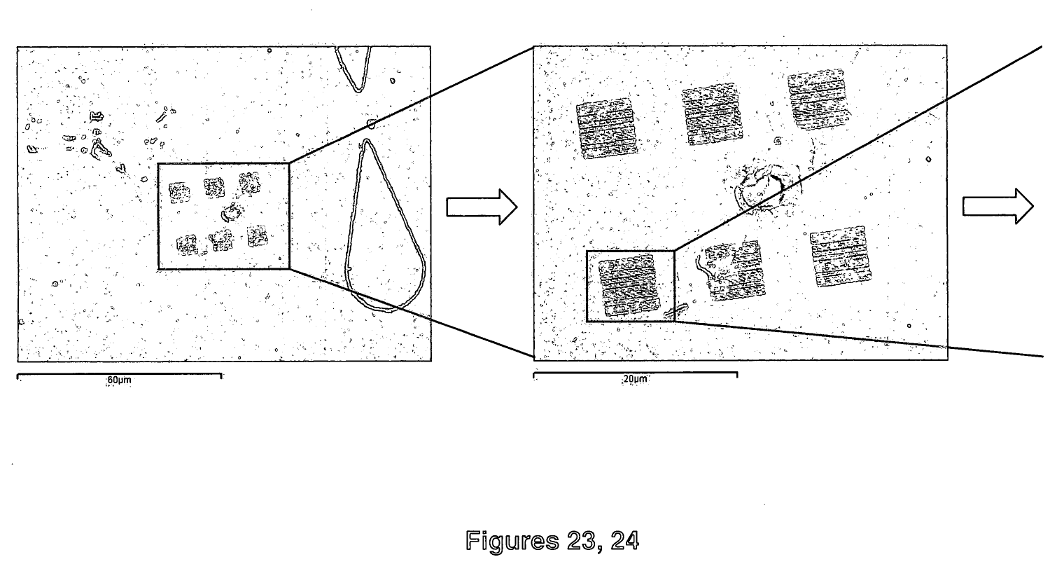

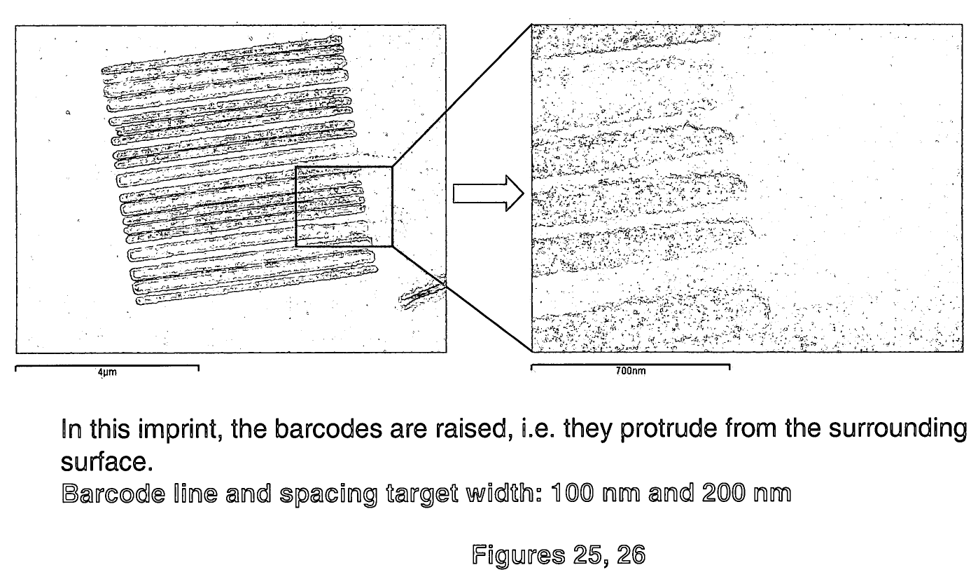

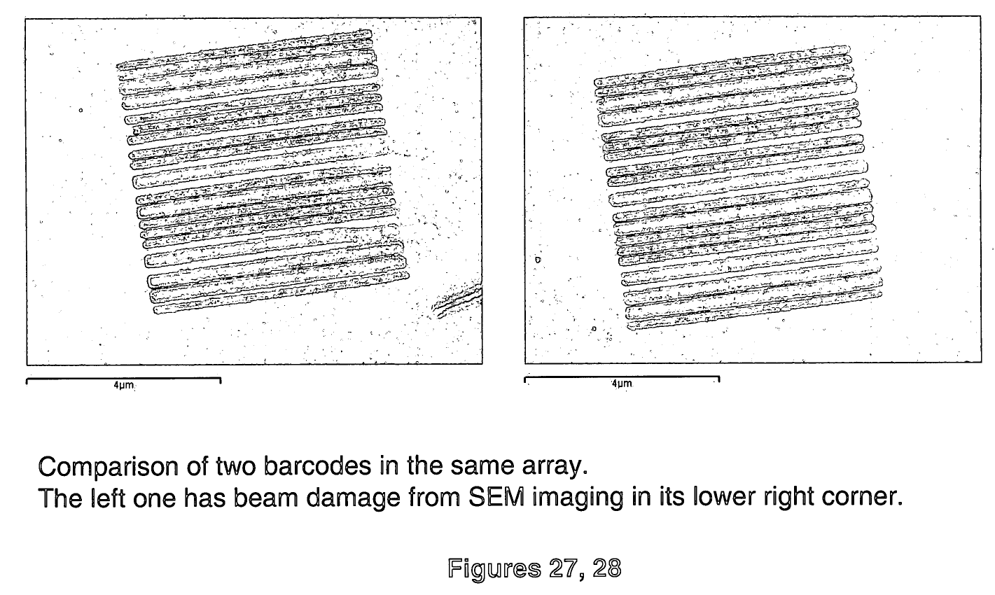

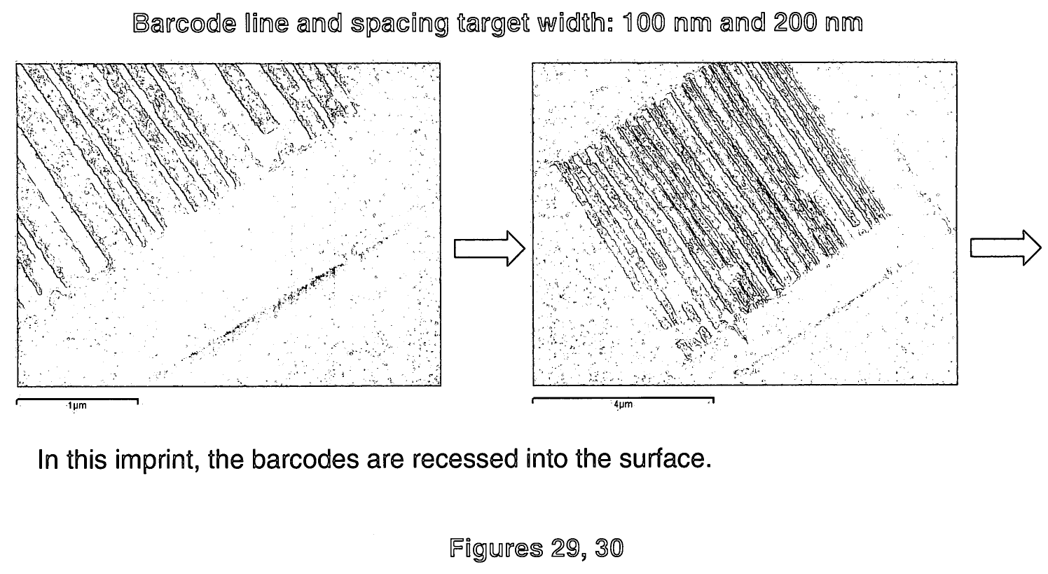

FIGS. 21-30 provide SEM images taken on a Hitachi S-4500 SEM instrument at 5 kV. The tablets were metal coated with 5 nm of Pt before imaging. Overt-covert imprinting is provided comprising a logo and bar codes close by.

In addition, a counterfeit, forged, or otherwise problematic pharmaceutical composition or other object can be fabricated without the actual identification features desired by for example a legitimate company, but potentially can have the correct identification features. One skilled in the art may not know, in some cases, whether the identification features are actually present until inspection. Hence, a plurality of unit pharmaceutical compositions or objects each having a surface to be inspected potentially can comprise identification features.

1. A method comprising:
providing a plurality of unit pharmaceutical compositions each having a surface to be inspected potentially comprising identification features;
providing an inspection device which provides the inspection zone which allows inspecting the surface of the unit pharmaceutical compositions potentially comprising identification features;
providing an introducing device which introduces the unit pharmaceutical composition into the inspection zone before inspection, wherein the unit pharmaceutical compositions are positioned and aligned so their surfaces are properly exposed for inspection;
providing a withdrawing device which withdraws the unit pharmaceutical compositions from the inspection zone after inspection;
operating the inspection device, the introducing device, and the withdrawing device so that an inspection rate of unit pharmaceutical compositions is achieved of at least about 10 units per hour.

2. The method according to claim 1, wherein the inspection rate is at least about 100 units per hour.

3. The method according to claim 1, wherein the inspection rate is at least about 1,000 units per hour.

4. The method according to claim 1, wherein the inspection rate is at least about 5,000 units per hour.

5. The method according to claim 1, wherein the inspection device comprises a scanning electron microscope.

6. The method according to claim 1, wherein the introducing device comprises a robotic device.

7. The method according to claim 1, wherein the withdrawing device comprises a robotic device.

8. The method according to claim 1, wherein the inspecting device is adapted to comprise a tray provided from the introducing device which holds a plurality of unit pharmaceutical compositions, wherein each unit is disposed in a separate region on the tray.

9. The method according to claim 1, wherein the identification features are present and comprise micron scale features of at least about one micron in lateral dimension.

10. The method according to claim 1, wherein the identification features are present and comprise nanometer scale features of less than about 1,000 nm in lateral dimension.

11. The method according to claim 1, wherein the identification features are present and comprise an overt feature.

12. The method according to claim 1, wherein the identification features are present and comprise a covert feature.

13. The method according to claim 1, wherein the identification features are present and comprise an overt feature and a covert feature.

14. The method according to claim 1, further comprising the step of identifying whether the unit pharmaceutical composition is genuine, fake, or illegally traded.

15. The method according to claim 1, wherein the inspection device comprises a high resolution device able to resolve features of less than about 1,000 nm.

16. The method according to claim 1, wherein said inspecting comprises laser scanning, optical microscopy, confocal microscopy or profiling, scanning ion microscopy, scanning electron microscopy, stylus or interferometric profiling, scanning probe microscopy, video scanning probe microscopy, or a combination thereof.

17. The method according to claim 1, wherein the introducing device comprises a computer controlled, automated device.

18. The method according to claim 1, wherein the withdrawing device comprises a computer controlled, automated device.

19. The method according to claim 1, wherein the introducing device comprises a tray which holds a plurality of unit pharmaceutical compositions wherein each unit is disposed in separate regions on the tray for inspection as the tray is robotically introduced into the inspection device.

20. (canceled)

21. The method according to claim 1, wherein operating the introducing device comprises placing and orienting the unit pharmaceutical compositions on a tray.

22. (canceled)

23. The method according to claim 1, wherein the operating of the inspection device comprises extracting a pattern image from the unit pharmaceutical compositions.

24. The method according to claim 1, wherein the operating of the inspection device comprises inspecting under vacuum.

25. The method according to claim 1, wherein operating the introducing device comprises introducing the unit pharmaceuticals into a vacuum.

26. (canceled)

27. The method according to claim 1, further comprising dispensing the unit pharmaceuticals onto a holding device for the unit pharmaceuticals, wherein one unit pharmaceutical is disposed in a holding zone on the holding device.

28. The method according to claim 1, wherein the inspecting device comprises a motorized stage for aligning the unit pharmaceutical compositions with the inspection device during inspection.

29. The method according to claim 1, wherein the inspection device is operated to carry out inspection in at least two stages.

30. The method according to claim 1, wherein the operation of the inspecting device comprises comparing a measured identification feature with a design feature to determine a match.

31. The method according to claim 1, wherein the method further comprises the steps of inspecting and decoding micrometer or nanometer scale identification features.

32. The method according to claim 1, wherein the operating steps are computer controlled.

33. The method according to claim 1, further comprising computer analysis of the results of operating the inspection device.

34. The method according to claim 1, wherein the unit pharmaceuticals comprise at least two types of identification features, one smaller and one larger, which are designed to be inspected by different inspection methods.

35. The method according to claim 1, wherein the unit pharmaceutical compositions are metal coated over the identification features.

36. The method according to claim 1, wherein the operation of the inspection device comprises a location step and an imaging step.

37. The method according to claim 1, wherein the inspection device comprises a high resolutions scanning electron microscope and multiple vacuum chambers.

38. The method according to claim 1, wherein the unit pharmaceutical compositions comprise tablets.

39. The method according to claim 1, further comprising the step of identifying the unit pharmaceutical with use of a pharmaceutical data base comprising geographical distribution.

40. The method according to claim 1, wherein the unit pharmaceuticals comprise a coating to improve inspection.

41. An apparatus comprising:
at least one scanning electron microscope (SEM) comprising a first vacuum chamber for SEM inspection, and at least one second vacuum chamber which can be evacuated and filled separately from the first vacuum chamber,
at least one first sample holder in the first vacuum chamber which is adapted to hold a plurality of sites for holding a plurality of unit pharmaceutical compositions to be inspected with the scanning electron microscope in the first vacuum chamber;
at least one second sample holder in the second vacuum chamber which is adapted to hold a plurality of sites for holding a plurality of unit pharmaceutical compositions to be inspected with the scanning electron microscope in the first vacuum chamber;
a sample holder transport device which transports the first sample holder from the first vacuum chamber to the second vacuum chamber; and the second sample holder from the second vacuum chamber to the first vacuum chamber,
wherein the apparatus is adapted for a sample inspection rate of at least about 10 units per hour.

42. The apparatus according to claim 41, wherein the apparatus is adapted for a sample inspection rate of at least about 100 units per hour.

43. The apparatus according to claim 41, wherein the apparatus is adapted for a sample inspection rate of at least about 1,000 units per hour.

44. The apparatus according to claim 41, wherein the apparatus is adapted for a sample inspection rate of at least about 5,000 units per hour.

45. The apparatus according to claim 41, further comprising a computer to control the SEM inspection.

46. The apparatus according to claim 41, further comprising a robot to control the sample holder transport device.

47. The apparatus according to claim 41, further comprising an alignment device to facilitate SEM inspection.

48. A pharmaceutical composition comprising:
a unit pharmaceutical composition comprising surface identification features which are adapted to be imaged by scanning electron microscopy;
metallic coating over the identification features adapted to improve the imaging by scanning electron microscopy.

49. The pharmaceutical composition according to claim 48, wherein the composition is a tablet or capsule.

50. The pharmaceutical composition according to claim 48, wherein the identification features are imprinted identification features.

51. A method for inspecting unit pharmaceutical compositions comprising:
providing a plurality of unit pharmaceutical compositions each having a surface to be inspected potentially comprising at least two identification features on each unit pharmaceutical;
providing a computer controlled scanning electron microscope inspection device which provides an inspection zone which allows inspecting the surface of the unit pharmaceutical compositions potentially comprising identification features;
providing an automated introducing device which introduces the unit pharmaceutical composition into the inspection zone before inspection;
providing an automated withdrawing device which withdraws the unit pharmaceutical composition from the inspection zone after inspection;
operating the inspection device, the introducing device, and the withdrawing device so that an inspection rate of unit pharmaceutical compositions is achieved of at least about 10 units per hour.

52. The method according to claim 51, wherein the inspection rate is at least about 100 units per hour.

53. The method according to claim 51, wherein the inspection rate is at least about 1,000 units per hour.

54. The method according to claim 51, wherein the inspection rate is at least about 5,000 units per hour.

55. The method according to claim 1, wherein the inspection device comprises a high resolution scanning electron microscope.

56. The method according to claim 51, wherein the introducing device comprises a robotic device.

57. The method according to claim 51, wherein the withdrawing device comprises a robotic device.

58. The method according to claim 51, wherein the inspecting device comprises a tray which holds a plurality of unit pharmaceutical compositions wherein each unit is disposed in separate regions on the tray for inspection.

59. The method according to claim 51, wherein the identification features comprise micron scale features of at least about one micron in lateral dimension.

60. The method according to claim 51, wherein the identification features comprise nanometer scale features of less than about 1,000 nm in lateral dimension.

61. The method according to claim 51, wherein the identification features comprise an overt feature.

62. The method according to claim 51, wherein the identification features comprise a covert feature.

63. The method according to claim 51, wherein the identification features comprise an overt feature and a covert feature.

64. The method according to claim 51, further comprising the step of identifying whether the unit pharmaceutical composition is genuine, fake, or illegally traded.

65. The method according to claim 51, wherein the inspection device comprises a high resolution scanning electron microscopy device able to resolve features of less than about 100 nm.

66. The method according to claim 51, wherein the inspection device comprises a scanning electron microscope adapted with a load lock.

67. The method according to claim 51, wherein the introducing device comprises a computer controlled, automated device synchronized with the inspecting device and the withdrawing device.

68. The method according to claim 51, wherein the withdrawing device comprises a computer controlled, automated device synchronized with the inspecting device and the introducing device.

69. The method according to claim 51, wherein the introducing device comprises a tray which holds a plurality of unit pharmaceutical compositions wherein each unit is disposed in separate regions on the tray for inspection.

70. The method according to claim 51, wherein the withdrawing device comprises a tray which holds a plurality of unit pharmaceutical compositions wherein each unit is disposed in separate regions on the tray for inspection.

71. The method according to claim 51, wherein operating the introducing device comprises placing and orienting the unit pharmaceutical compositions on a tray.

72. The method according to claim 51, wherein operating the introducing device comprises aligning the unit pharmaceutical compositions disposed on a tray.

73. The method according to claim 51, wherein the operating of the inspection device comprises extracting a pattern image from the unit pharmaceutical compositions.

74. The method according to claim 51, wherein the operating of the inspection device comprises inspecting under high vacuum.

75. The method according to claim 51, wherein operating the introducing device comprises introducing the unit pharmaceuticals into a vacuum.

76. The method according to claim 51, wherein operating the withdrawing device comprises withdrawing the unit pharmaceuticals from a vacuum.

77. The method according to claim 51, wherein the introducing device comprises a dispensing device which dispenses unit pharmaceuticals onto a holding device for the unit pharmaceuticals, wherein one unit pharmaceutical is disposed in one holding zone on the holding device.

78. The method according to claim 51, wherein the inspecting device comprises a motorized stage for aligning the unit pharmaceutical compositions with the inspection device during inspection.

79. The method according to claim 51, wherein the inspection device is operated to carry out inspection in two stages, one stage being an SEM stage and another stage being a non-SEM stage.

80. The method according to claim 51, wherein the operation of the inspecting device comprises comparing a measured identification feature with a design feature to determine a match.

81. The method according to claim 51, wherein the method is used for inspecting and decoding nanometer scale identification features.

82. The method according to claim 51, wherein the method is used for inspecting and decoding micrometer scale identification features.

83. The method according to claim 51, further comprising computer analysis of the results of operating the inspection device.

84. The method according to claim 51, wherein the unit pharmaceuticals comprise at least two types of identification features, one smaller and one larger, which are designed to be inspected by different inspection methods.

85. The method according to claim 51, wherein the unit pharmaceutical compositions are metal coated over the identification features.

86. The method according to claim 51, wherein the operation of the inspection device comprises a location step and an imaging step for the identification features.

87. The method according to claim 51, wherein the inspection device comprises a high resolutions scanning electron microscope and multiple vacuum chambers.

88. The method according to claim 51, wherein the unit pharmaceutical compositions comprise tablets.

89. The method according to claim 51, wherein the method is computer controlled and automated with robots.

90. The method according to claim 51, wherein the unit pharmaceuticals comprise a coating to improve inspection.

91. A method for inspecting unit pharmaceutical compositions in high throughput mode with scanning electron microscopy comprising the combination of steps:
providing a plurality of unit pharmaceutical compositions each having a surface to be inspected potentially comprising at least two identification features on each unit pharmaceutical and adapted for SEM inspection;
providing a computer controlled scanning electron microscope inspection device which provides an inspection zone which allows inspecting the surface of the unit pharmaceutical compositions potentially comprising identification features;
providing an automated, robotic introducing device which introduces the unit pharmaceutical composition into the inspection zone before inspection;
providing an automated, robotic withdrawing device which withdraws the unit pharmaceutical composition from the inspection zone after inspection;
operating the inspection device, the introducing device, and the withdrawing device so that an inspection rate of unit pharmaceutical compositions is achieved of at least about 10 units per hour;
processing inspection data collected from the inspection device to determine whether identification features are present or absent.

92. A method comprising:
providing a plurality of unit compositions or objects each having a surface to be inspected potentially comprising identification features;
providing an inspection device which provides the inspection zone which allows inspecting the surface of the unit compositions or objects potentially comprising identification features; wherein the unit compositions or objects are positioned and aligned so their surfaces are properly exposed for inspection providing an introducing device which introduces the unit composition or object into the inspection zone before inspection;
providing a withdrawing device which withdraws the unit composition or object from the inspection zone after inspection;
operating the inspection device, the introducing device, and the withdrawing device so that an inspection rate of unit compositions or objects is achieved of at least about 10 units per hour.

93. A method comprising:
providing a plurality of unit pharmaceutical compositions each having a surface to be inspected comprising identification features;
providing an inspection device which provides the inspection zone which allows inspecting the surface of the unit pharmaceutical compositions comprising identification features;
providing an introducing device which introduces the unit pharmaceutical composition into the inspection zone before inspection; wherein the unit pharmaceutical compositions are positioned and aligned so their surfaces are properly exposed for inspection,
providing a withdrawing device which withdraws the unit pharmaceutical composition from the inspection zone after inspection;
operating the inspection device, the introducing device, and the withdrawing device so that an inspection rate of unit pharmaceutical compositions is achieved of at least about 10 units per hour.
